(19) United States
(12) Patent Application Publication
(10) Pub. No.: US 2020/0283351 A1
(43) Pub. Date: Sep. 10, 2020

(54) HALOGENATED HETEROALKENYL- AND HETEROALKYL-FUNCTIONALIZED ORGANIC COMPOUNDS AND METHODS FOR PREPARING SUCH COMPOUNDS

(71) Applicant: Arkema Inc., King of Prussia, PA (US)

(72) Inventors: Benjamin Bin CHEN, Wayne, PA (US); Robert G. SYVRET, Allentown, PA (US); Craig A. POLSZ, Newtown, PA (US); Haiming LIU, Upper Umchlan, PA (US); Jay Fingeret MILLER, Downingtown, PA (US); Lucy M. CLARKSON, Chadds Ford, PA (US)

(21) Appl. No.: 16/651,386

(22) PCT Filed: Sep. 25, 2018
(86) PCT No.: PCT/US2018/052553

§ 371 Date: Mar. 27, 2020

Related U.S. Application Data
(60) Provisional application No. 62/563,736, filed on Sep. 27, 2017.

(51) Int. Cl. C07B 39/00; C07C 41/16; C07C 303/22; C07D 233/58; C07D 231/12; C07C 213/06; C07D 317/22; C07C 67/04; C07C 43/247; C07C 69/653

(52) U.S. Cl. CPC C07B 39/00; C07C 41/16; C07C 303/22; C07D 233/58; C07C 69/653; C07C 213/06; C07D 317/22; C07C 67/04; C07C 43/247; C07D 231/12

(57) ABSTRACT

A method for synthesizing halogenated organic compounds, such as halogenated alkenyl group-containing and halogenated alkyl group-containing compounds having a heteroatom (e.g., O,N.S) coupled to a carbon atom of a halogenated alkenyl or halogenated alkyl group, involves reacting a halogenated olefin such as a chloro-substituted trifluoropropenyl compound with an active hydrogen-containing organic compound such as an alcohol (e.g., an aliphatic monoalcohol, aliphatic polyalcohol, or a phenolic compound), a primary amine, a secondary amine or a thiol.

### FIELD OF THE INVENTION

The present invention relates to synthetic methods for introducing halogenated heteroalkenyl-containing functional groups (e.g., trifluoropropenyl-containing functional groups, including trifluoropropenylether, trifluoropropenylthioether and trifluoropropenylamine substituents) and halogenated heteroalkyl-containing functional groups into organic compounds, as well as the reaction products obtained by such methods.

### BACKGROUND OF THE RELATED ART

The preparation of new organic compounds containing various types of substituents, particularly substituents containing functional groups capable of further reaction (e.g., polymerization or derivatization) or imparting desired physical or chemical properties to an organic compound, continues to be of great interest in the chemical, advanced materials (including electronics materials and coatings), agricultural and pharmaceutical industries. As is well known, substituted or functionalized organic compounds may have a number of different end uses, such as solvents, monomers, synthetic intermediates, active pharmaceutical ingredients, pesticides, herbicides, complexing agents and so forth.

One type of organic substituent of particular potential interest would be a substituent containing a halogenated (e.g., fluorinated) alkenyl or halogenated alkyl moiety, such as a trifluoropropenyl moiety (e.g., a trifluoroprop-1-enyl moiety, corresponding to the structure F₃C—CH═CH—, or a trifluoroprop-2-enyl moiety, corresponding to the structure F₃C—C(—)═CH₂). Also of interest would be a substituent having a heteroatom (O, N, S) coupled to a halogenated (e.g., fluorinated) alkenyl moiety or halogenated (e.g., fluorinated) alkyl moiety. Illustrative substituents of this type include trifluoropropenylether, trifluoropropenylthioether, and trifluoropropenylamine substituents. For example, such a substituent may correspond to the structure F₃C—CH═CH—X— (in cis or trans form) or F₃C—C(—X—)═CH₂, where X is O (oxygen), S (sulfur) or NR (where N is nitrogen and R is hydrogen or an organic moiety such as an optionally substituted alkyl group).

### BRIEF SUMMARY OF THE INVENTION

Various non-limiting aspects of the invention may be summarized as follows: Aspect 1: A method of making a halogenated organic compound, comprising reacting an active hydrogen-containing organic compound selected from the group consisting of alcohols, primary amines, secondary amines and thiols with a halogenated olefin containing a carbon-carbon double bond, wherein at least one carbon of the carbon-carbon double bond is substituted with at least one substituent selected from the group consisting of halogens and halogenated alkyl groups, to produce the halogenated organic compound.

Aspect 2: The method of Aspect 1, wherein the halogenated olefin contains one, two, three, four or more fluorine atoms.

Aspect 3: The method of Aspect 1 or 2, wherein the halogenated organic compound is a halogenated heteroalkenyl-functionalized organic compound (e.g., a fluorinated heteroalkenyl-functionalized organic compound, a chlorinated heteroalkenyl-functionalized organic compound, or a chlorinated/fluorinated heteroalkenyl-functionalized organic compound).

Aspect 4: The method of Aspect 1 or 2, wherein the halogenated organic compound is a halogenated heteroalkyl-functionalized organic compound (e.g., a fluorinated heteroalkyl-functionalized organic compound, a chlorinated heteroalkyl-functionalized organic compound, or a chlorinated/fluorinated heteroalkyl-functionalized organic compound).

Aspect 5: The method of any of Aspects 1 to 4, wherein the halogenated olefin has a fluorinated alkyl group substituted on one carbon of the carbon-carbon double bond.

Aspect 6: The method of any of Aspects 1 to 5, wherein the halogenated olefin has a perfluorinated alkyl group substituted on one carbon of the carbon-carbon double bond.

Aspect 7: The method of any of Aspects 1 to 6, wherein the halogenated olefin has a structure in accordance with formula (1):

CX¹X²═CX³X⁴ (1)

wherein X¹, X², X³ and X⁴ are independently selected from the group consisting of hydrogen (H), chlorine (Cl), fluorine (F), bromine (Br), iodine (I) and halogenated and non-halogenated C1-C20 alkyl groups, subject to the proviso that one or more of X¹, X², X³ and X⁴ is selected from the group consisting of chlorine (Cl), fluorine (F), bromine (Br), iodine (I) and halogenated alkyl groups. In other aspects, none of X¹, X², X³ or X⁴ is Br, in particular when the active hydrogen-containing compound is an aliphatic alcohol. In other aspects, at least one of X¹, X², X³ or X⁴ is Cl and the halogenated olefin additionally contains one, two, three, four or more fluorine atoms.

Aspect 8: The method of any of Aspects 1 to 7, wherein the halogenated olefin is selected from the group consisting of CClF═CH₂ (VCF), CH₂═CF₂ (VDF), CFH═CH₂, CF₂═CHF, CF₃CF═CH₂, CF₂═CF₂ (TFE), CH₂═CHCl, CHCl═CHCl, CH₂═CCl₂, CF₂═CFCl; CF₂═CHCl, CF₃CCl═CH₂, CF₃CCl═CClH, CF₃CH═CCl₂, CF₃CF═CCl₂, CF₃CF═CClH, CF₃CCl═CFH, CF₃CCl═CF₂, CF₃CCl═CFCl, CF₃CF═CFCl, CF₃CH═CHCl, CF₃CF═CFH, CF₃CH═CF₂, CF₃CF═CF₂, CF₃CH₂CF═CH₂, CF₃CH═CFCH₃, CF₃CF═CHCF₃, CF₃CCl═CHCF₃, CF₂HCH₂CF═CH₂, CF₂HCH₂CF═CHCl and CF₂HCH═CFCH₂Cl.

Aspect 9: The method of any of Aspects 1 to 8, wherein the halogenated olefin is reacted with a phenolic compound.

Aspect 10: The method of any of Aspects 1 to 8, wherein the halogenated olefin is reacted with an aliphatic alcohol.

Aspect 11: The method of any of Aspects 1 to 8, wherein the halogenated olefin is reacted with an aliphatic polyalcohol (polyol).

Aspect 12: The method of any of Aspects 1 to 8, wherein the halogenated olefin is reacted with a masked aliphatic polyalcohol which is an aliphatic polyol having a plurality of hydroxyl groups wherein at least one hydroxyl group is blocked and at least one hydroxyl group is a free hydroxyl group.

Aspect 13: The method of any of Aspects 1 to 8, wherein the halogenated olefin is reacted with a primary or secondary amine.

Aspect 14: The method of any of Aspects 1 to 8, wherein the halogenated olefin is reacted with a thiol.

Aspect 15: The method of any of Aspects 1 to 14, wherein the reacting is carried out under basic conditions.

Aspect 16: The method of any of Aspects 1 to 15, wherein the reacting is carried out in the presence of an inorganic base.

Aspect 17: The method of Aspect 16, wherein the inorganic base is selected from the group consisting of alkali metal hydroxides and alkali metal salts of carbonic acid.

Aspect 18: The method of any of Aspects 1 to 17, wherein the reacting is carried out in a liquid medium.

Aspect 19: The method of Aspect 18, wherein the liquid medium is comprised of one or more organic solvents.

Aspect 20: The method of Aspect 19, wherein the one or more organic solvents are selected from the group consisting of polar, non-protic organic solvents.

Aspect 21: The method of Aspect 19, wherein the one or more organic solvents are polar, non-protic organic solvents having dielectric constants of from 2 to 190.

Aspect 22: The method of any of Aspects 1 to 21, wherein the reacting is carried out in the presence of a phase transfer catalyst.

Aspect 23: The method of Aspect 9, wherein the phenolic compound has structure Ar(OH)ₓ, wherein Ar is an optionally substituted aromatic moiety and x is an integer of 1 or more.

Aspect 24: The method of Aspect 23, wherein x is 1, 2 or 3.

Aspect 25: The method of Aspect 23 or 24, wherein Ar is selected from the group consisting of optionally substituted phenyl groups, optionally substituted naphthyl groups, and optionally substituted anthryl groups.

Aspect 26: The method of any of Aspects 23 to 25, wherein Ar is an aromatic moiety substituted with one or more substituents selected from the group consisting of halogen, optionally substituted alkyl, optionally substituted heteroalkyl, optionally substituted cycloalkyl, optionally substituted heterocycloalkyl, optionally substituted alkoxy, optionally substituted aroxy, optionally substituted aryl, optionally substituted heteroaryl, cyano, optionally substituted carboxyl, sulfate, nitrile, and nitro.

Aspect 27: The method of any of Aspects 1 to 26, wherein the active hydrogen-containing organic compound and the halogenated olefin are reacted at a temperature of from about 5° C. to about 200° C. or about 20° C. to about 120° C. for a time of from about 0.5 hours to about 120 hours.

Aspect 28: The method of any of Aspects 1 to 27, wherein the active hydrogen-containing organic compound and the halogenated olefin are reacted in a stoichiometric ratio of (moles active hydrogen-containing organic compound)/x:moles halogenated olefin, wherein x=number of active hydrogens per molecule of the active hydrogen-containing organic compound, of from about 1:8 to about 8:1.

Aspect 29: A trifluoropropenylether-substituted aromatic compound of formula (I):

Ar(OCR¹═CHR²)ₓ (I)

wherein Ar is an optionally substituted aromatic moiety, x is an integer of 1 or more, and

either R¹ is CF₃ and R² is H or R¹ is H and R² is CF₃.

Aspect 30: The trifluoropropenylether-substituted aromatic compound of Aspect 29, wherein x is 1, 2 or 3.

Aspect 31: The trifluoropropenylether-substituted aromatic compound of Aspect 29 or 30, wherein Ar is selected from the group consisting of optionally substituted phenyl groups, optionally substituted naphthyl groups, and optionally substituted anthryl groups.

Aspect 32: The trifluoropropenylether-substituted aromatic compound of any of Aspects 29 to 31, wherein Ar is an aromatic moiety substituted with one or more substituents selected from the group consisting of halogen, alkyl, cyano, sulfate, nitrile and nitro.

Aspect 33: The trifluoropropenylether-substituted aromatic compound of any of Aspects 29 to 32, wherein the trifluoropropenylether-substituted aromatic compound is selected from the group consisting of 4-chlorophenyl-3,3-3-trifluoropropenyl ether, 1,4-bis(3,3,3-trifluoropropenyloxy)benzene, 4-fluorophenyl-3,3,3-trifluoropropenyl ether, 4-methylphenyl-3,3,3-trifluoropropenyl ether, 3-cyanophenyl-3,3,3-trifluoropropenyl ether, 2-fluorophenyl-3,3,3-trifluoropropenyl ether, 3-nitrophenyl-3,3,3-trifluoropropenyl ether, 2,4-dichlorophenyl-3,3,3-trifluoropropenyl ether, 2-chloro-4-fluorophenyl-3,3,3-trifluoropropenyl ether, 4-(3,3,3-trifluoropropenyl)phenyl sulfate, 4-fluorophenyl-3,3,3-trifluoroprop-2-enyl ether, 3-nitrophenyl-3,3,3-trifluoroprop-2-enyl ether, 2-fluorophenyl-3,3,3-trifluoroprop-2-enyl ether, 4-methylphenyl-3,3,3-trifluoroprop-2-enyl ether, 4-chlorophenyl-3,3,3-trifluoroprop-2-enyl ether, 3-cyanophenyl-3,3,3-trifluoroprop-2-enyl ether, 1,4-bis(3,3,3-trifluoroprop-2-enyl)phenyl ether, 2,4-dichlorophenyl-3,3,3-trifluoroprop-2-enyl ether, 2-chloro-4-fluorophenyl-3,3,3-trifluoroprop-2-enyl ether, and 4-(3,3,3-trifluoroprop-2-enyl)phenyl sulfate, sodium salt.

Aspect 34: A haloalkyl ether (meth)acrylate corresponding to general structure (I):

X¹X²HC—CX³X⁴—O—R—O—C(═O)—CR¹═CH₂ (I)

wherein R is an organic moiety, X¹, X², X³ and X⁴ are independently selected from hydrogen, halogen, alkyl or haloalkyl, subject to the proviso that at least one of X¹, X², X³ or X⁴ is halogen or a haloalkyl group, and R¹ is hydrogen or methyl or fluorine.

Aspect 35: The haloalkyl ether (meth)acrylate of Aspect 34, wherein at least two of X¹, X², X³ or X⁴ are selected from the group consisting of halogens and haloalkyl groups.

Aspect 36: The haloalkyl ether (meth)acrylate of Aspect 34 or 35, wherein at least two of X¹, X², X³ or X⁴ are selected from the group consisting of fluorine and fluoroalkyl groups.

Aspect 37: The haloalkyl ether (meth)acrylate of any of Aspects 34 to 36, wherein at least one of X¹, X², X³ or X⁴ is fluorine or a fluoroalkyl group.

Aspect 38: The haloalkyl ether (meth)acrylate of any of Aspects 34 to 37, wherein each of X¹, X², X³ and X⁴ is halogen or a haloalkyl group.

Aspect 39: The haloalkyl ether (meth)acrylate of any of Aspects 34 to 38, wherein one of X¹, X², X³ or X⁴ is a C1-C8 haloalkyl group.

Aspect 40: The haloalkyl ether (meth)acrylate of any of Aspects 34 to 39, wherein one of X¹, X², X³ or X⁴ is a C1-C8 fluoroalkyl group.

Aspect 41: The haloalkyl ether (meth)acrylate of Aspect 34, wherein a) X¹ is chlorine and X², X³ and X⁴ are fluorine or b) X³ is chlorine and X¹, X² and X⁴ are fluorine.

Aspect 42: The haloalkyl ether (meth)acrylate of any of Aspects 34 to 41, wherein R is an alkylene segment or a poly(oxyalkylene) segment.

Aspect 43: The haloalkyl ether (meth)acrylate of any of Aspects 34 to 42, wherein R is an ethylene segment or a poly(oxyethylene) segment.

Aspect 44: The haloalkyl ether (meth)acrylate of any of Aspects 34 to 43, wherein R is —[CH₂CH₂O]ₙ—CH₂CH₂— and n is 0 or an integer of from 1 to 10.

Aspect 45: The haloalkyl ether (meth)acrylate of any of Aspects 34 to 44, wherein the moiety X¹X²HC—CX³X⁴—O—R—O— has a molecular weight not greater than 900 daltons.

Aspect 46: The haloalkyl ether (meth)acrylate of any of Aspects 34 to 45, wherein R is a non-halogenated organic moiety.

Aspect 47: The haloalkyl ether (meth)acrylate of any of Aspects 34 to 46, wherein R is an aliphatic organic moiety, optionally containing one or more oxygen atoms.

Aspect 48: The haloalkyl ether (meth)acrylate of any of Aspects 34 to 47, wherein R is a saturated aliphatic organic moiety, optionally containing one or more ether oxygen atoms.

Aspect 49: A haloalkenyl ether (meth)acrylate corresponding to general structure (II):

X¹X²C═CX³—O—R—O—C(═O)—CR¹═CH₂ (II)

wherein R is an organic moiety, X¹, X², X³ and are independently selected from hydrogen, halogen, alkyl or haloalkyl, subject to the proviso that at least one of X¹, X², or X³ is halogen or a haloalkyl group, and R¹ is hydrogen or methyl or fluorine.

Aspect 50: The haloalkyl ether (meth)acrylate of Aspect 49, wherein at least two of X¹, X², X³ or X⁴ are selected from the group consisting of halogens and haloalkyl groups.

Aspect 51: The haloalkyl ether (meth)acrylate of Aspect 49 or 50, wherein at least two of X¹, X², X³ or X⁴ are selected from the group consisting of fluorine and fluoroalkyl groups.

Aspect 52: The haloalkyl ether (meth)acrylate of any of Aspects 49 to 51, wherein at least one of X¹, X², X³ or X⁴ is fluorine or a fluoroalkyl group.

Aspect 53: The haloalkyl ether (meth)acrylate of any of Aspects 49 to 52, wherein each of X¹, X², X³ and X⁴ is halogen or a haloalkyl group.

Aspect 54: The haloalkyl ether (meth)acrylate of any of Aspects 49 to 53, wherein one of X¹, X², X³ or X⁴ is a C1-C8 haloalkyl group.

Aspect 55: The haloalkyl ether (meth)acrylate of any of Aspects 49 to 54, wherein one of X¹, X², X³ or X⁴ is a C1-C8 fluoroalkyl group.

Aspect 56: The haloalkyl ether (meth)acrylate of Aspect 49, wherein a) X¹ is chlorine and X², X³ and X⁴ are fluorine or b) X³ is chlorine and X¹, X² and X⁴ are fluorine.

Aspect 57: The haloalkyl ether (meth)acrylate of any of Aspects 49 to 56, wherein R is an alkylene segment or a poly(oxyalkylene) segment.

Aspect 58: The haloalkyl ether (meth)acrylate of any of Aspects 49 to 57, wherein R is an ethylene segment or a poly(oxyethylene) segment.

Aspect 59: The haloalkyl ether (meth)acrylate of any of Aspects 49 to 58, wherein R is —[CH₂CH₂O]ₙ—CH₂CH₂— and n is 0 or an integer of from 1 to 10.

Aspect 60: The haloalkyl ether (meth)acrylate of any of Aspects 49 to 59, wherein the moiety X¹X²HC—CX³X⁴—O—R—O— has a molecular weight not greater than 900 daltons.

Aspect 61: The haloalkyl ether (meth)acrylate of any of Aspects 49 to 60, wherein R is a non-halogenated organic moiety.

Aspect 62: The haloalkyl ether (meth)acrylate of any of Aspects 49 to 61, wherein R is an aliphatic organic moiety, optionally containing one or more oxygen atoms.

In certain aspects of the invention, the synthesis of trifluoropropenylether-containing compounds may be accomplished by reacting the appropriate alcohol with a base in the presence of 1-chloro-3,3,3-trifluoro-prop-1-ene, hereafter referred to as 1233zd. The alcohol may be an aliphatic alcohol (e.g., an aliphatic monoalcohol or an aliphatic polyalcohol) or an aromatic alcohol (e.g., a phenolic compound). A representative example for the preparation of a substituted-phenyl-3,3,3-trifluoropropenyl ether is provided in the general equation (1) below (where X can be hydrogen or a substituent such as halo, alkyl, alkoxy, cyano, sulfate, nitrile, or nitro):

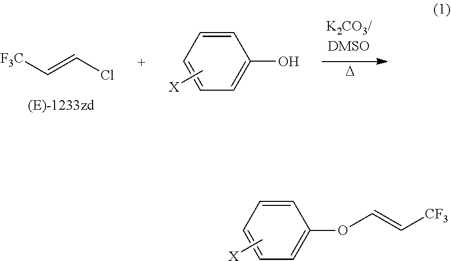

In this general example, reaction of a substituted phenol, X—ArOH, with trans-(E)-1233zd in the presence of potassium carbonate (K₂CO₃) in DMSO solvent at elevated temperature provides the substituted phenyl 3,3,3-trifluoropropenyl ether, X—Ar—O—CH═CH—CF₃, where Ar=phenyl. Cis-(Z)-1233zd is also an equally effective source of the trifluoropropenyl moiety.

In another aspect of this invention, 2-chloro-3,3,3-trifluoroprop-1-ene is used as the source of the trifluoropropene moiety and the product is the corresponding 3,3,3-trifluoroprop-2-ene phenyl ether. A representative example is provided below in general equation (2):

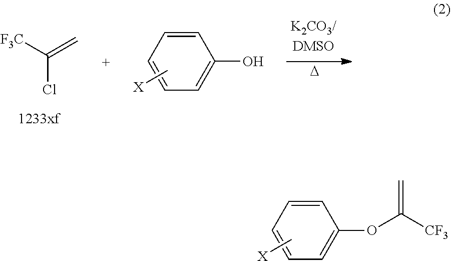

Halogenated heteroalkenyl- and halogenated heteroalkyl-functionalized organic compounds prepared in accordance with the methods of the present invention are useful in a number of applications, including as synthetic intermediates and monomers (in cases where the halogenated compound comprises at least one functional group, such as a vinyl group, a vinylidene group, a (meth)acrylate group or an active hydrogen-containing functional group (e.g., hydroxyl) capable of participating in a curing or polymerization reaction to form a polymer). Such uses are described in more detail in the provisional United States applications being filed simultaneously herewith under Attorney Docket Nos. IR 4328A, IR 4328B and IR 4328C, the entire disclosures of each of which are incorporated herein by reference for all purposes.

### DETAILED DESCRIPTION OF CERTAIN ASPECTS OF THE INVENTION

### Halogenated Olefins

The methods of the present invention employ a halogenated olefin (for example, a fluorinated olefin) as a reactant. As used herein, the term “halogenated olefin” refers to an organic compound containing at least one carbon-carbon double bond and at least one halogen atom (Cl, F, Br, I). As used herein, the term “fluorinated olefin” refers to an organic compound containing at least one carbon-carbon double bond and at least one fluorine atom (and optionally one or more halogen atoms other than fluorine, in particular one or more chlorine atoms).

The halogenated olefin may contain one, two, three or more halogen atoms, such as bromine, chlorine, fluorine or iodine atoms or combinations thereof (e.g., at least one fluorine atom and at least one chlorine atom). In certain embodiments, the halogenated olefin contains at least one halogen atom substituted on at least one of the carbon atoms involved in a carbon-carbon double bond present in the halogenated olefin. However, in other embodiments, the halogenated olefin does not contain any halogen atom attached to either of the carbon atoms involved in the carbon-carbon double bond, but does contain at least one halogenated alkyl group substituted on at least one of the carbon atoms involved in the carbon-carbon double bond. Suitable fluorinated olefins include olefins containing one, two, three or more fluorine (F) atoms. The fluorine atom(s) may be substituted on one or both of the carbon atoms involved in a carbon-carbon double bond and/or may be present as a substituent on a moiety, such as an alkyl group, that is attached to one or both of the carbon atoms involved in a carbon-carbon double bond. For example, the fluorinated olefin may comprise one or more fluoroalkyl (e.g., perfluoroalkyl) groups, such as fluoromethyl, difluoromethyl, trifluoromethyl, fluoroethyl, difluoroethyl, trifluoroethyl, tetrafluoroethyl, perfluoroethyl, fluoropropyl, difluoropropyl, trifluoropropyl, tetrafluoropropyl, pentafluoropropyl, hexafluoropropyl, perfluoropropyl and the like and analogues thereof wherein wherein a portion of the fluorine atoms and/or one or more of the hydrogen atoms are replaced with other halogen atoms (e.g., Cl). The fluorinated olefin may comprise one or more halogen atoms other than fluorine, in particular one or more chlorine (Cl), iodine (I) and/or bromine (Br) atoms. In certain embodiments of the invention, the halogenated olefin or fluorinated olefin may comprise at least one chlorine atom substituted on a carbon atom involved in a carbon-carbon double bond. In further embodiments of the invention, the halogenated olefin or fluorinated olefin may comprise at least one hydrogen atom substituted on a carbon atom involved in a carbon-carbon double bond. For example, fluoroolefins, hydrofluoroolefins, chloroolefins, hydrochloroolefins, chlorofluoroolefins, and hydrochlorofluoroolefins may all be employed as the halogenated olefin reactant in the present invention. Suitable types of fluorinated olefins include fluoroethylenes, chlorofluoroethylenes, fluoropropenes, chlorofluoropropenes, fluorobutenes, chlorofluorobutenes, fluoropentenes, chlorofluoropentenes, fluorohexenes, chloro-fluorohexenes, and the like. Other suitable fluorinated olefins are cyclo-fluorobutenes, cyclo-chlorofluorobutenes, cyclo-fluoropentenes, cyclo-chlorofluoropentenes, cyclo-fluorohaxenes, and cyclo-chlorofluorohaxenes, such as 1-chloro-2,3,3-trifluorocyclobutene, 1,2-dichlorotetrafluorocyclobutene, hexafluorocyclobutene, 1H-heptafluorocyclopentene, 1-chloro-3,3,4,4,5,5-hexafluorocyclopentene, 1-chloroheptafluorocyclopentene, octafluorocyclopentene, 1,2-dichlorohexafluorocyclopentene, 1,2,3-trichloropentafluorocyclopentene, perfluorocyclohexene, 1,2-dichlorooctafluorocyclohexene, 1H-perfouorocyclohexene, and the like. In various embodiments of the invention, the halogenated olefin comprises two, three, four, five, six or more carbon atoms, e.g., 2-20 carbon atoms, 2-8 carbon atoms, 2-6 carbon atoms or 2-4 carbon atoms.

According to certain aspects of the invention, the halogenated olefin may have a structure in accordance with formula (1):

CX¹X²═CX³X⁴ (1)

wherein X¹, X², X³ and X⁴ are independently selected from the group consisting of hydrogen (H), chlorine (Cl), fluorine (F), bromine (Br), iodine (I) and halogenated and non-halogenated C1-C20 alkyl groups, subject to the proviso that one or more of X¹, X², X³ and X⁴ is selected from the group consisting of chlorine (Cl), fluorine (F), bromine (Br), iodine (I) and halogenated alkyl groups (e.g., C1-C20 halogenated alkyl groups, in particular halogenated alkyl groups containing one, two, three, four or more halogen atoms, in particular F and/or Cl, such as chlorinated alkyl groups, fluorinated alkyl groups, such as trifluoromethyl, and chlorinated/fluorinated alkyl groups). In one embodiment, the halogenated olefin does not contain a bromine atom substituted on a carbon atom involved in a carbon-carbon double bond. In other embodiments, the halogenated olefin contains a bromine atom substituted on a carbon atom involved in a carbon-carbon double bond and contains one or more halogen atoms other than the bromine atom substituted on a carbon atom involved in a carbon-carbon double bond.

Specific representative examples of halogenated olefins suitable for use in the present invention include, but are not limited to:

CClF═CH₂ (sometimes referred to as VCF)
CH₂═CF₂ (sometimes referred as VDF)

### CFH═CH₂

### CF₂═CHF

CF₃CF═CH₂
CF₂═CF₂ (sometimes referred to as TFE)

### CF₂═CHCl

CF₃CCl═CH₂

### CF₃CH═CHCl

### CF₃CF═CFH

CF₃CH═CF₂
CF₃CF═CF₂
CF₃CH₂CF═CH₂
CF₃CH═CFCH₃
CF₃CF═CHCF₃
CF₃CCl═CHCF₃
CF₂HCH₂CF═CH₂
CF₂HCH₂CF═CHCl
CF₂HCH═CFCH₂Cl

### CH₂═CHCl

### CHCl═CHCl

CH₂═CCl₂

### CF₂═CFCl;

CF₃CCl═CH₂

### CF₃CCl═CClH

CF₃CH═CCl₂
CF₃CF═CCl₂

### CF₃CF═CFCl

### CF₃CF═CClH

### CF₃CCl═CFH

CF₃CCl═CF₂

### CF₃CCl═CFCl

All possible isomers (e.g., E or Z isomers) of the above-mentioned halogenated olefins can be used.

In one embodiment, a chloro-substituted trifluoropropenyl compound is employed as the halogenated olefin. Suitable chloro-substituted trifluoropropenyl compounds include 1-chloro-3,3,3-trifluoro-prop-1-ene (also known as 1233zd) and 2-chloro-3,3,3-trifluoroprop-1-ene. Either the cis or trans isomer of 1-chloro-3,3,3-trifluoro-prop-1-ene may be used (i.e., trans-(E)-1233zd or cis-(Z)-1233zd).

In various embodiments of the invention, the halogenated olefin reactant may have a purity (as calculated in weight percent) of at least 80, at least 85, at least 90, at least 95, at least 99 or even 100%. Methods of preparing and purifying such halogenated olefins are well known in the art. In addition, suitable halogenated olefins are available from commercial sources, such as The Arkema Group.

### Active Hydrogen-Containing Organic Compounds

The active hydrogen-containing organic compound utilized in the methods of the present invention may be selected from the group consisting of alcohols, primary amines, secondary amines, and thiols. The active hydrogen-containing organic compound may comprise one or more active hydrogens per molecule (e.g., one, two, three, four, five or more active hydrogens per molecule). Such active hydrogens may be in the form of hydroxyl groups (—OH), thiol groups (—SH) and/or primary or secondary amine groups (—NH₂ or —NH—, wherein each open bond is to a carbon atom). It is understood that under certain reaction conditions (for example, when the reaction is catalyzed or promoted by a base), the active hydrogen-containing organic compound may be present in deprotonated or partially deprotonated form (e.g., —O⁻, —S⁻). The active hydrogen-containing organic compound may be monomeric, oligomeric or polymeric. There is no particular known restriction with respect to the number of carbon atoms which may be present in the active hydrogen-containing organic compound, but in various embodiments of the invention the active hydrogen-containing organic compound may be comprised of from 1 to 30 or from 2 to 20 carbon atoms.

The term “alcohol” refers to any organic compound bearing at least one hydroxyl group (—OH) substituted on an organic moiety. The term “thiol” refers to any organic compound bearing at least one thiol group (—SH) substituted on an organic moiety. The term “primary amine” refers to any organic compound bearing at least one —NH₂ group substituted on an organic moiety. The term “secondary amine” refers to any organic compound containing, as a substituent on an organic moiety or as part of a cyclic organic structure, at least one —NH— group (wherein the nitrogen atom is bonded to two carbon atoms).

The organic moiety portion of the active hydrogen-containing organic compound is not limited and may be, for example, an optionally substituted alkyl group, an optionally substituted heteroalkyl group, an optionally substituted alkylene group, an optionally substituted heteroalkylene group, an optionally substituted aryl group, an optionally substituted heteroaryl group, an optionally substituted cycloalkyl group, or an optionally substituted heterocycloalkyl group.

As used herein, the term “alkyl” is defined to include saturated aliphatic hydrocarbons including straight (linear) chains and branched chains. In some embodiments, the alkyl group has 1 to 20 carbon atoms, 1 to 10 carbon atoms, 1 to 6 carbon atoms, or 1 to 4 carbon atoms. An alkyl group optionally can be substituted by one or more (e.g. 1 to 5) suitable substituents. Heteroatoms such as oxygen, sulfur, phosphorus and nitrogen (in the form of tertiary amine moieties) may be present in the alkyl group, to provide a heteroalkyl group (e.g., an alkyl group containing one or more ether, thioether, or amino linkages). Illustrative examples of heteroalkyl groups include —CH₂CH₂N(CH₃)₂ and —CH₂CH₂OCH₂CH₃.

As used herein, the term “alkenyl” refers to aliphatic hydrocarbons having at least one carbon-carbon double bond, including straight chains and branched chains having at least one carbon-carbon double bond. In some embodiments, the alkenyl group has 2 to 20 carbon atoms, 2 to 10 carbon atoms, 2 to 6 carbon atoms, 3 to 6 carbon atoms, or 2 to 4 carbon atoms. An alkenyl group optionally can be substituted by one or more (e.g. 1 to 5) suitable substituents. When the active hydrogen-containing organic compound contains an alkenyl group, the alkenyl group may exist as the pure E form, the pure Z form, or any mixture thereof. Heteroatoms such as oxygen, sulfur and nitrogen (in the form of tertiary amine moieties) may be present in the alkylene group, to provide a heteroalkylene group (e.g., an alkylene group containing one or more ether, thioether, or amino linkages).

As used herein, the term “cycloalkyl” refers to saturated or unsaturated, non-aromatic, monocyclic or polycyclic (such as bicyclic) hydrocarbon rings (e.g., monocyclics such as cyclopropyl, cyclobutyl, cyclopentyl, cyclohexyl, cycloheptyl, cyclooctyl, cyclononyl, or bicyclics including spiro, fused, or bridged systems. The cycloalkyl group may have 3 to 15 carbon atoms. In some embodiments the cycloalkyl may optionally contain one, two or more non-cumulative non-aromatic double or triple bonds and/or one to three oxo groups. Also included in the definition of cycloalkyl are moieties that have one or more aromatic rings (including aryl and heteroaryl) fused to the cycloalkyl ring. The cycloalkyl group optionally can be substituted by 1 or more (e.g., 1 to 5) suitable substituents.

As used herein, the term “aryl” refers to all-carbon monocyclic or fused-ring polycyclic aromatic groups having a conjugated pi-electron system. The aryl group may, for example, have 6, 10 or 14 carbon atoms in the ring(s). Phenyl, naphthyl and anthryl are example of suitable aryl groups. Some examples of compounds containing one or more aryl groups are 4-(2-acryloxyethoxy)-2-hydroxybenzophenone, 3-allyl-4-hydroxyacetophenone, and 4-methacryloxy-2-hydroxybenzophenone. The aryl group optionally can be substituted by 1 or more (e.g., 1 to 5) suitable substituents.

As used herein, the term “heteroaryl” refers to monocyclic or fused-ring polycyclic aromatic heterocyclic groups with one or more heteroatom ring members (ring-forming atoms) each independently selected from O, S and N in at least one ring. The heteroaryl group may have 5 to 14 ring-forming atoms, including 1 to 13 carbon atoms, and 1 to 8 heteroatoms selected from O, S, and N. A heteroaryl group optionally can be substituted by 1 or more (e.g., 1 to 5) suitable substituents.

As used herein, the term “heterocycloalkyl” refers to a monocyclic or polycyclic [including 2 or more rings that are fused together, including spiro, fused, or bridged systems, for example, a bicyclic ring system], saturated or unsaturated, non-aromatic 4- to 15-membered ring system, including 1 to 14 ring-forming carbon atoms and 1 to 10 ring-forming heteroatoms each independently selected from O, S and N. The heterocycloalkyl group optionally can be substituted by 1 or more (e.g., 1 to 5) suitable substituents.

Suitable types of groups which may be present as substituents in any of the above-mentioned organic moieties include one or more of the following: halo (F, Cl, Br, I), alkyl, aryl, alkoxy, cyano (—CN), carboxyl (—C(═O)R, where R is an organo substituent such as alkyl, aryl or the like), carboxylic acid (—C(═O)OH, cycloalkoxy, aryloxy, tertiary amino, sulfate (—SO₃M, wherein M is alkali metal or ammonium), oxo, nitrile and the like.

As used herein, the term “halo” or “halogen” group is defined to include fluorine, chlorine, bromine or iodine.

As used herein, the term “alkoxy” refers to an —O-alkyl group. The alkoxy group optionally can be substituted by 1 or more (e.g., 1 to 5) suitable substituents.

As used herein, the term “cycloalkoxy” or “cycloalkyloxy” refers to an —O-cycloalkyl group. The cycloalkoxy or cycloalkyloxy group optionally can be substituted by 1 or more (e.g., 1 to 5) suitable substituents.

As used here, the term “aryloxy” refers to an —O-aryl group. An example of an aryloxy group is —O-phenyl [i.e., phenoxy]. The aryloxy group optionally can be substituted by 1 or more (e.g., 1 to 5) suitable substituents.

As used herein, the term “oxo” refers to ═O. When an oxo is substituted on a carbon atom, they together form a carbonyl moiety [—C(═O)—]. When an oxo is substituted on a sulfur atom, they together form a sulfinyl moiety [—S(═O)—]; when two oxo groups are substituted on a sulfur atom, they together form a sulfonyl moiety [—S(═O)₂—].

As used herein, the term “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group (up to that every hydrogen atom on the designated atom or moiety is replaced with a selection from the indicated substituent group), provided that the normal valency of the designated atom or moiety is not exceeded, and that the substitution results in a stable compound. For example, if a phenyl group (i.e., —C₆H₅) is optionally substituted, then up to five hydrogen atoms on the phenyl ring can be replaced with substituent groups.

In certain embodiments of the invention, the active hydrogen-containing organic compound corresponds to the general structure Q(YH)ₓ, wherein Q is a substituted or unsubstituted organic moiety (e.g., alkyl, heteroalkyl, cycloalkyl, heterocycloalkyl, aryl, heteroaryl and substituted variants thereof), Y is O, S or NR (where R is H or a substituted or unsubstituted organic moiety, such as an optionally substituted alkyl group), and x is an integer of 1 or more (e.g., 1-10, 1-5 or 1-3). In such compounds, the oxygen, sulfur or nitrogen atom of each Y moiety is bonded to a carbon atom of Q. Where x is an integer of 2 or more, the Y moieties may be the same as or different from each other.

For example, the active hydrogen-containing organic compound may correspond to the general formula:

Ar(OH)ₓ

wherein Ar is a substituted or unsubstituted aromatic moiety and x is an integer of 1 or more (e.g., 1, 2 or 3). In certain embodiments, Ar is selected from the group consisting of phenyl, substituted phenyl groups, naphthyl, substituted naphthyl groups, anthryl, and substituted anthryl groups. Ar may be an aromatic moiety, such as a phenyl group, substituted with one or more substituents at any position of the aromatic ring(s); the substituents may, for example, be selected from the group consisting of halogen, alkyl, cyano, sulfate and nitro, but any of the additional above-mentioned types of substituents could be used (alone or in combination).

In other embodiments of the invention, the active hydrogen-containing organic compound may be an aliphatic polyalcohol, that is, an aliphatic alcohol containing two or more hydroxyl groups per molecule (e.g., two to six hydroxyl groups per molecule), which are sometimes referred to as “polyols”. By controlling the reaction conditions (e.g., the stoichiometry of the aliphatic polyalcohol and the halogenated olefin), all of the hydroxyl groups may be reacted or only a portion of the hydroxyl groups may be reacted. The partially reacted products may be of interest when it is desired to obtain a product that contains at least one halogenated (e.g., fluorinated) alkenyl or alkyl group, but also at least one hydroxyl group that is still available for further reaction (such as with a hydroxyl-reactive compound other than a halogenated olefin, such as an isocyanate or a carboxylic acid or anhydride) or that can participate in hydrogen bonding or the like (thereby varying the properties of the product). Examples of suitable aliphatic polyalcohols include, but are not limited to, C₂-C₁₈ aliphatic diols (including glycols and oligomeric glycols such as diethylene glycol and triethylene glycol), sugars, sugar alcohols, glycerol, pentaerythritol, aliphatic triols (e.g., trihydroxybutanes and trihydroxypentanes), trimethylolpropane, trimethylolethane, dipentaerythritol and alkoxylated derivatives thereof (e.g., where any of the aforementioned aliphatic polyalcohols has been reacted with from 1 to 750 (e.g., 1 to 30) moles of an alkylene oxide such as ethylene oxide and/or propylene oxide per mole of aliphatic polyalcohol).

According to other aspects of the invention, the active hydrogen-containing organic compound may contain two or more active hydrogen-containing functional groups, wherein at least one active hydrogen-containing functional group is masked/blocked and at least one active hydrogen-containing functional group remains in unprotected form and can participate in the desired reaction with a halogenated olefin. Alternatively, the unprotected active hydrogen-containing functional group may first be reacted with another reactant to yield an intermediate containing an active hydrogen (e.g., in the form of a hydroxyl group) which is then reacted with a halogenated olefin. For example, the unprotected active hydrogen-containing functional group may be reacted with one or more equivalents of an alkylene oxide (e.g., ethylene oxide, propylene oxide) to form an alkoxylated intermediate containing a hydroxyl group, which is thereafter reacted with a halogenated olefin.

Following such reaction(s), the masked/blocked active hydrogen-containing functional group(s) can be optionally deprotected, thereby generating at least one active hydrogen-containing functional group. Any of the methods known in the art for removing a masking or blocking group may be utilized. For example, an acetal or ketal protective group can be removed by treatment of the intermediate with aqueous acid. As another example, a benzyl protective group may be removed using hydrogenation.

The regenerated active hydrogen-containing functional group(s) can, if so desired, be further reacted, for example with a reactant containing at least one functional group that is reactive with the active hydrogen-containing functional group(s). In other embodiments, at least one active hydrogen-containing functional group is masked/blocked so as to introduce a reactive functional group (e.g., a (meth)acrylate, (meth)acrylamide or allyl functional group) into the active hydrogen-containing organic compound. The reactive functional group can be left in place following reaction with the halogenated olefin, so as to provide, for example, a site capable of being cured or polymerized such as a (meth)acrylate, (meth)acrylamide or allyl group.

Non-limiting examples of masked/blocked polyols include compounds such as (2,2-dimethyl-1,3-dioxolan-4-yl)methanol (also known as solketal), 4-hydroxymethyl-1,3-dioxolan-2-one (also known as glycerin carbonate), hydroxyethyl methacrylate (HEMA), poly ethoxy ethyl methacrylates, such as HEMA-10, 2-hydroxyethyl acrylate or HEA, hydroxypolyethoxy allyl ether, such as hydroxypolyethoxy (10) allyl ether, hydroxypropyl methacrylate, hydroxypropyl acrylate, 3-phenoxy-2-hydroxy propyl methacrylate, pentaerythritol triacrylate, poly(propylene glycol) methacrylate, such as poly(propylene glycol) 300 methacrylate, 1,1,1-trimethylolpropane diallyl ether (pure or mono/di/triallyl mixture), 1,1,1-trimethylolpropane mono-ethers, glycerol monomethacrylate, N-(2-hydroxypropyl)methacrylamide, hydroxypolyethoxy allyl ether, sodium 1-allyloxy-2-hydroxypropyl sulfonate, N-hydroxyethyl acrylamide, and polyols partially esterified with a carboxylic acid.

In particular embodiments of the invention, an aliphatic polyalcohol is employed in which one or more of the hydroxyl groups are masked or blocked, with one or more of the hydroxyl groups remaining free for reaction with halogenated olefin. Once the blocked/masked polyalcohol has been reacted with the halogenated olefin, the blocking/masking group(s) (sometimes also referred to as protecting groups) may be optionally removed so as to generate one or more free hydroxyl groups. Any of the blocking or masking reagents or techniques known in the field of organic chemistry to be suitable for masking hydroxyl groups may be employed in the present invention. Typically, however, it will be desirable to employ a blocking or masking group that remains stable (i.e., is not removed to any significant extent) under the conditions used to react the masked aliphatic polyalcohol with the halogenated olefin. For example, if a basic catalyst is employed during the masked aliphatic polyalcohol/halogenated olefin reaction, the blocking/masking group(s) should be resistant to deblocking or demasking under such basic conditions. Illustrative examples of suitable blocking/masking groups include, but are not limited to, silyl ether groups, acetal groups, ketal groups, benzyl groups and the like. Solketal is a particular example of a blocked/masked aliphatic polyalcohol, wherein two hydroxyl groups of glycerol are blocked through a ketal group, with the other hydroxyl group being free to react with a halogenated olefin in accordance with the present invention. Other examples of suitable protecting groups for hydroxyl functional groups include, but are not limited to, acetyl (Ac), benzoyl (Bz), beta-methoxyethoxymethylether (MEM), dimethoxytrityl (DMT), methoxymethyl ether (MOM), methoxytrityl (MMT), p-methoxybenzyl ether (PMB), methylthiomethyl ether, pivaloyl (Piv), tetrahydropyranyl (THP), tetrahydrofuryl (THF), trityl (triphenylmethyl, Tr), silyl ether, methyl ether, tertiary alkyl ether and ethoxyethyl ether (EE). In one embodiment of the invention, an aliphatic polyalcohol containing three or more hydroxyl groups per molecule is reacted with an aldehyde or ketone to form an acetal or ketal, wherein two of the hydroxyl groups of the aliphatic polyalcohol separated by two or three carbon atoms have reacted with the aldehyde or ketone to form a cyclic acetal- or ketal-containing structure.

Similarly, other types of active hydrogen-containing functional groups (e.g., thiol groups, primary amine groups, secondary amine groups) which may be present in the active hydrogen-containing compound employed as a starting material in the present invention may be masked or blocked using any of the masking/blocking techniques known in the art. One or more non-blocked active hydrogen-containing functional groups which are still present may then be reacted with the halogenated olefin to obtain a halogenated intermediate, which may then be de-blocked (unmasked) to provide one or more active hydrogen-containing functional groups (which are then available for further reaction or derivatization). Suitable amine protecting groups include, for example, carbobenzyloxy (Cbz) groups, p-methoxybenzyl carbonyl (Moz or MeOZ) groups, tert-butyloxycarbonyl (BOC) groups, 9-fluorenylmethyloxycarbonyl (FMOC) groups), acetyl (Ac) groups, benzoyl (Bz) groups, benzyl (Bn) groups, carbamate groups, p-methoxybenzyl (PMB) groups, 3,4-dimethoxybenzyl (DMPM) groups, p-methoxyphenyl (PMP) groups, tosyl (Ts) groups, trichloroethyl chloroformate (Troc) groups, sulfonamide (e.g., Nosyl and Nps) groups and the like.

Specific suitable phenolic compounds which may be used as a starting material include the phenolic compounds mentioned in the Examples. Specific suitable secondary amines which may be used as a starting material include the secondary amines mentioned in the Examples. Amino acids are also useful as the active hydrogen-containing organic compound.

Following reaction with a halogenated olefin, one or more of the active hydrogens of the active hydrogen-containing organic compound (i.e., one or more of the hydrogens in one or more of the —YH moieties) is replaced by an alkenyl or alkyl group (e.g., —CF═CH₂, —CF₂CFHCF₃, —CF₂CFClH. —CF₂CClH₂, —CF₂CF₂H, —CH═CHCF₃ or —C(CF₃)═CH₂). In certain embodiments, all of the active hydrogens of the active hydrogen-containing organic compound are replaced by an alkenyl or alkyl group. In such embodiments, the halogenated organic compound obtained may be represented by the general structure Q(Y-Alk)ₙ wherein Q, Y and n have the same meaning as stated above and Alk is a halogenated alkenyl or halogenated alkyl group. In other embodiments (where n=2 or more), less than all of the active hydrogens of the active hydrogen-containing organic compound are replaced by a halogenated alkenyl or halogenated alkyl group. In such embodiments, the halogenated organic compound obtained may be represented by the general structure Q(YH)ₙ₋ₘ(Y-Alk)ₘ wherein Q, Y and n have the same meaning as stated above, m is an integer of from 1 to n−1, and Alk is a halogenated alkenyl or halogenated alkyl group. In embodiments where Y is NH, the hydrogen atom of NH may or may not be similarly replaced by a halogenated alkenyl or alkyl group.

Without wishing to be bound by theory, it is believed that the reaction of the present invention proceeds by addition of the active hydrogen-containing functional group of the active hydrogen-containing organic compound across the double bond of the halogenated olefin. Such reaction forms a halogenated alkyl group (i.e., the halogenated olefin is converted to a halogenated alkyl group which is present within the product formed). Typically, the heteroatom of the active hydrogen-containing functional group (e.g., the oxygen atom of a hydroxyl group) becomes preferably bonded to the more “halogen heavy” carbon atom of the carbons involved in the carbon-carbon double bond of the halogenated olefin (i.e., the carbon having the greatest number of halogen atoms bonded to it). In certain cases, mixtures of different products are obtained, wherein the heteroatom of the active hydrogen-containing functional group becomes bonded to each of the carbon atoms involved in the carbon-carbon double bond. An alkenyl group results from elimination of hydrohalide from the halogenated alkyl group. Such elimination may be favored by increasing the basicity of the reaction medium.

The aforementioned transformations may be generically illustrated as follows.

R—OH+ZXC=CZ₂→(R—O—)ZXC—CHZ₂ Initial reaction:

(R—O—)ZXC—CHZ₂→(R—O—)ZC=CZ₂+HX Elimination:

R=organic moiety (e.g., alkyl, aryl)

X=halogen (e.g., F, Cl)

Z=hydrogen, halogenated or non-halogenated organic moiety, halogen

The present invention makes possible, for example, the preparation of trifluoropropenylether-substituted aromatic compounds of formula (I):

Ar(OCR¹═CHR²)ₓ (I)

wherein Ar is a substituted or unsubstituted aromatic moiety, x is an integer of 1 or more (e.g., 1, 2 or 3), and either R¹ is CF₃ and R² is H or R¹ is H and R² is CF₃.

Ar may be selected from the group consisting of phenyl, substituted phenyl, naphthyl, substituted naphthyl, anthryl, and substituted anthryl. Ar may be an aromatic moiety, such as phenyl, substituted with one or more substituents, such as one or more substituents selected from the group consisting of halogen, alkyl, alkoxy, cyano, sulfate and nitro. Such substituent or substituents may be attached to any of the carbon atoms of the aromatic ring(s), other than the carbon atom(s) bonded to the oxygen atom(s) of the trifluoropropenyl group(s). Where Ar is a substituted phenyl group and x=1, for example, a substituent may be present at the 2, 3, 4, 5 and/or 6 position of the phenyl ring (the trifluoropropenyl group being present at the 1 position of the phenyl ring).

Specific examples of halogenated organic compounds capable of being produced in accordance with the present invention include, but are not limited to, halogenated organic compounds which are the reaction product of an active hydrogen-containing organic compound selected from the group consisting of solketal, glycerine carbonate, aminoethanol, hydroxyethyl acrylate, hydroxyethyl methacrylate, poly ethoxy ethyl methacrylate, hydroxypropyl methacrylate, pentaerythritol triacrylate, N-(2-hydroxypropyl)methacrylamide, and glycerol monomethacrylate with a halogenated olefin selected from the group consisting of CF₂═CH₂, CFCl═CH₂, CF₂═CHCl, CF₂═CFCl, CF₂═CF₂, CF₃CF═CF₂, CF₃CF═CH₂, CF₃CH═CFH, CF₃CCl═CH₂, and CF₃CH═CHCl.

Specific examples of trifluoropropenylether-substituted aromatic compounds in accordance with the present invention include, but are not limited to: 4-chlorophenyl-3,3-3-trifluoropropenyl ether, 1,4-bis(3,3,3-trifluoropropenyloxy)benzene, 4-fluorophenyl-3,3,3-trifluoropropenyl ether, 4-methylphenyl-3,3,3-trifluoropropenyl ether, 3-cyanophenyl-3,3,3-trifluoropropenyl ether, 2-fluorophenyl-3,3,3-trifluoropropenyl ether, 3-nitrophenyl-3,3,3-trifluoropropenyl ether, 2,4-dichlorophenyl-3,3,3-trifluoropropenyl ether, 2-chloro-4-fluorophenyl-3,3,3-trifluoropropenyl ether, 4-(3,3,3-trifluoropropenyl)phenyl sulfate, 4-fluorophenyl-3,3,3-trifluoroprop-2-enyl ether, 3-nitrophenyl-3,3,3-trifluoroprop-2-enyl ether, 2-fluorophenyl-3,3,3-trifluoroprop-2-enyl ether, 4-methylphenyl-3,3,3-trifluoroprop-2-enyl ether, 4-chlorophenyl-3,3,3-trifluoroprop-2-enyl ether, 3-cyanophenyl-3,3,3-trifluoroprop-2-enyl ether, 1,4-bis(3,3,3-trifluoroprop-2-enyl)phenyl ether, 2,4-dichlorophenyl-3,3,3-trifluoroprop-2-enyl ether, 2-chloro-4-fluorophenyl-3,3,3-trifluoroprop-2-enyl ether, and 4-(3,3,3-trifluoroprop-2-enyl)phenyl sulfate, sodium salt.

The methods of the present invention are also useful for the synthesis of haloalkyl ether (meth)acrylates and haloalkylene ether (meth)acrylates. Haloalkyl (meth)acrylates may be characterized as organic compounds which comprise a haloalkyl moiety bonded through an ether linkage and an organic spacer moiety (in that sequence) to a (meth)acrylate functional group. Haloalkenyl ether (meth)acrylates may be characterized as organic compounds which comprise a haloalkenyl moiety bonded through an ether linkage and an organic spacer moiety (in that sequence) to a (meth)acrylate functional group. Haloalkyl ether (meth)acrylates and haloalkenyl ether (meth)acrylates may sometimes be collectively referred to herein as “haloalkyl/haloalkenyl ether (meth)acrylates”. As used herein, the term “(meth)acrylate” refers to acrylate (—C(═O)CH═CH₂) and methacrylate (—C(═O)C(CH₃)═CH₂) functional groups. As used herein, the term “haloalkyl” refers to an alkyl group which is substituted with one or more halogen atoms, which may be the same as or different from each other if more than one halogen atom is present. As used herein, the term “haloalkenyl” refers to an alkenyl group which is substituted with one or more halogen atoms, which may be the same as or different from each other if more than one halogen atom is present. Where the haloalkyl or haloalkenyl group contains two or more carbon atoms, halogen(s) may be substituted on any or all of the carbon atoms. An individual carbon atom in the haloalkyl or haloalkenyl group may be substituted with one, two or three halogen atoms, which may be the same as or different from each other. In addition to halogen, individual carbon atoms within the haloalkyl or haloalkenyl group may be substituted with one or more hydrogen atoms. Where the haloalkyl or haloalkenyl group contains two or more carbon atoms, one or more carbon atoms may be non-halogenated, provided that at least one carbon atom is halogenated. As used herein, the term “alkyl” means a paraffinic hydrocarbon group which may be derived from an alkane by dropping one hydrogen from the formula, such as ethyl (CH₃CH₂—). As used herein, the term “alkenyl” refers to an unsaturated hydrocarbon group having at least one carbon-carbon double bond which may be derived from an alkene by dropping one hydrogen from the formula, such as propenyl (CH₃CH═CH— or CH₂═C(CH₃)—). The term halogen, as used herein, means fluorine (F), chlorine (Cl), bromine (BR) or iodine (I).

In certain embodiments, the haloalkyl ether (meth)acrylate corresponds to general structure (I)

X¹X²HC—CX³X⁴—O—R—O—C(═O)—CR¹═CH₂ (I)

wherein R is an organic moiety, X¹, X², X³ and X⁴ are independently selected from hydrogen, halogen or haloalkyl, subject to the proviso that at least one of X¹, X², X³ or X⁴ is halogen or a haloalkyl group, and R¹ is hydrogen or methyl. According to certain embodiments of the invention, at least two of X¹, X², X³ or X⁴ are selected from the group consisting of halogens and haloalkyl groups. At least two of X¹, X², X³ or X⁴ are selected from the group consisting of fluorine and fluoroalkyl groups, in certain embodiments. In other embodiments, at least one of X¹, X², X³ or X⁴ is fluorine or a fluoroalkyl group. Each of X¹, X², X³ and X⁴ is halogen or a haloalkyl group, according to other embodiments of the invention. One of X¹, X², X³ or X⁴ may be a C1-C8 haloalkyl group, in particular a C1-C8 fluoroalkyl group such as a C1-C8 perfluoroalkyl group (e.g., trifluoromethyl).

In other embodiments, the haloalkenyl ether (meth)acrylate corresponds to general structure (IA):

X¹X²C═CX³—O—R—O—C(═O)—CR¹═CH₂ (IA)

wherein R is an organic moiety, X¹, X² and X³ are independently selected from hydrogen, halogen or haloalkyl, subject to the proviso that at least one of X¹, X² or X³ is halogen or a haloalkyl group, and R¹ is hydrogen or methyl. According to certain embodiments of the invention, at least two of X¹, X² or X³ are selected from the group consisting of halogens and haloalkyl groups. At least two of X¹, X² or X³ are selected from the group consisting of fluorine and fluoroalkyl groups, in certain embodiments. In other embodiments, at least one of X¹, X² or X³ is fluorine or a fluoroalkyl group. Each of X¹, X² and X³ is halogen or a haloalkyl group, according to other embodiments of the invention. One of X¹, X² or X³ may be a C1-C8 haloalkyl group, in particular a C1-C8 fluoroalkyl group such as a C1-C8 perfluoroalkyl group (e.g., trifluoromethyl).

Illustrative examples of suitable haloalkyl ether moieties include, without limitation:

CH₃—CF₂—O—

### CH₃—CFH—O—

CH₂F—CF₂—O—
CF₃CF(CH₃)—O—
CF₂H—CF₂—O—
CH₂Cl—CF₂—O—
CH₃C(CF₃)Cl—O—
CH₂Cl—CH(CF₃)—O—
CFH₂—CF(CF₃)—O—
CF₃CH₂—CF₂—O—
CF₃CFH—CF₂—O—
CH₃—CF(CH₂CF₃)—O—
CF₃—CH₂—CF(CH₃)—O—
CF₃—CH₂—CF(CF₃)—O—
CF₃—CH₂—CCl(CF₃)—O—
CH₃CF(CH₂CF₂H)—O—
CH₂Cl—CF(CH₂CF₂H)—O—
CF₂H—CH₂—CF(CH₂Cl)—O—

### CH₃CHCl—O—

### CH₂Cl—CHCl—O—

CH₃CCl₂—O—

### CFClH—CF₂—O—

CH₃—CCl(CF₃)—O—
CClH₂—CCl(CF₃)—O—
CF₃—CH₂—CCl₂—O—
CCl₂H—CF(CF₃)—O—

### CFClH—CF(CF₃)—O—

CClH₂—CF(CF₃)—O—
CFH₂—CCl(CF₃)—O—
CF₃—CHCl—CF₂—O—

### CF₃—CHCl—CFCl—O—

Illustrative examples of suitable haloalkenyl ether moieties include, without limitation, moieties analogous to the above-mentioned haloalkyl ether moieties, but where hydrohalide has been eliminated to form a carbon-carbon double bond between the carbon bonded to the ether oxygen and the adjacent carbon atom. The haloalkyl ether (meth)acrylate may correspond to general structure (I) wherein a) X¹ is chlorine and X², X³ and X⁴ are fluorine or b) X³ is chlorine and X¹, X² and X⁴ are fluorine.

R may be an alkylene segment or a poly(oxyalkylene) segment, in certain aspects of the invention. As used herein, the term “alkylene” means a paraffinic hydrocarbon group which may be derived from an alkane by dropping two hydrogens from the formula, such as ethylene (—CH₂CH₂—). The term “oxyalkylene” means an alkylene group coupled to an ether oxygen, as in oxyethylene for example (—CH₂CH₂O—). Thus, in various aspects of the invention, a haloalkyl/haloalkenyl ether (meth)acrylate corresponding to general structure (I) or (IA) is provided wherein R is an ethylene segment or a poly(oxyethylene) segment. For example, R may be —[CH₂CH₂O]ₙ—CH₂CH₂— wherein n is 0 or an integer of from 1 to 10 or higher. Although R may be a substituted or heteroatom-containing organic moiety, such as an oxygen-containing organic moiety, in certain embodiments R is non-halogenated (i.e., does not contain any halogen atoms). R may be, for example, aliphatic (including straight chain or branched aliphatic or cycloaliphatic), aromatic, or contain both aliphatic and aromatic structural units, but in certain embodiments is aliphatic and does not contain any aromatic structural units. In particular, R may be a saturated aliphatic organic moiety, optionally containing one or more oxygen atoms such as ether oxygen atoms (oxygen atoms forming an ether linkage).

The moiety X¹X²HC—CX³X⁴—O—R—O— or X¹X²C═CX³—O—R—O—, according to certain embodiments, may have a molecular weight not greater than 900 daltons, not greater than 800 daltons or not greater than 700 daltons.

The methods of the present invention are also useful for the synthesis of haloalkyl diether according to general structure of X¹X²HC—CX³X⁴OCH₂CH₂OX³X⁴C—CX¹X²H wherein X¹, X², X³ and X⁴ are fluorine or chlorine. Haloalkyl diethers according to the present invention are generally electrochemically stable, therefore suitable as a solvents and/or additives for Li batteries utilizing LiPF₆, LiTFSI, LiFSI, LiTDI, and other lithium-sulfurs. Examples of haloalkyl diethers according to the present invention are FClHC—CF₂—O—CH₂CH₂—O—CF₂CFClH, HF₂C—CF₂—O—CH₂CH₂—OCF₂—CF₂H, and the like.

### Reaction Conditions

The active hydrogen-containing organic compound and the halogenated olefin are contacted with each other for a time and at a temperature effective to achieve the desired extent of reaction between the starting materials, whereby the desired halogenated organic compound is produced.

The reaction may be carried out using any suitable manner and any suitable equipment, apparatus or system, which may vary depending upon the reactants and the reaction conditions selected. For example, the reaction may be performed in a batch, continuous, semi-continuous or staged or step-wise mode. Where one or more of the reactants is relatively volatile (e.g., where the reactant has a boiling point less than or only somewhat above the desired reaction temperature), it may be advantageous to conduct the reaction in a closed or pressurized vessel and/or to provide a means to collect any of the volatile reactant that may distill out of the reaction mixture (using, for example, a reflux condenser) and return such reactant to the reaction mixture. The reaction vessel may be provided with suitable heating, cooling and/or stirring/agitation means, as well as lines for introducing and/or withdrawing materials.

In one embodiment of the invention, the reaction is carried out under elevated pressure, i.e., pressures greater than atmospheric pressure. For example, pressures of from ambient to 50 bar may be utilized.

The active hydrogen-containing organic compound and the halogenated olefin may be reacted neat. An excess of one of the reactants may be utilized, in effect, as a solvent. In another embodiments, a reaction medium such as a solvent or combination of solvents may be employed to solubilize or otherwise disperse the reactants and/or the reaction product(s). According to certain aspects of the invention, one or more organic solvents are employed in admixture with the reactants. In particular, polar, non-protic organic solvents may be utilized, such as sulfoxides (e.g., DMSO), amides (e.g., dimethyl formamide (DMF), dimethylacetamide, diethylacetamide, hexamethylphosphoramide (HMPA), hexametylphosphorous triamide (HMPT)), nitriles (e.g., acetonitrile, benzonitrile), sulfolane, esters (e.g., ethyl acetate), ethers (THF), N-methyl-2-pyrrolidinone (NMP), nitrobenzene, nitromethane, ketones (e.g., acetone, methylethylketone), carbonates such as 4-fluoro-1,3-dioxolan-2-one (FEC), cis-4,5-difluoro-1,3-dioxolan-2-one (cis-DFEC), trans-4,5-difluoro-1,3-dioxolan-2-one (trans-DFEC), 4,4-difluoro-1,3-dioxolan-2-one (gem-DFEC), 4-fluoromethyl-1,3-dioxolan-2-one (FPC), 4-trifluoromethyl-1,3-dioxolan-2-one (TFPC), ethylene carbonate (EC), propylene carbonate (PC), trans-butylene carbonate (t-BC), dimethyl carbonate (DMC) and the like and combinations thereof. Polar, protic solvents such as alcohols and aminoalcohols (e.g., 2-aminoethanol) may also be used under at least certain reaction conditions, for example, where the active hydrogen-containing organic compound is more reactive than the polar, protic solvent with the halogenated olefin. An organic solvent or a mixture of organic solvents having a dielectric constant between 2 and 190 under ambient conditions (25° C.), preferably between 4 and 120, and even more preferably between 13 and 92 may be employed in the present invention. Water may also be present, provided that the desired product is not readily converted to undesirable products in the presence of water, in combination with one or more organic solvents (which may be miscible with water or immiscible with water). For example, when potassium hydroxide is used, water content is preferably less than about 24 wt %, more preferably less than about 15 wt %, even more preferably less than about 10 wt %. Accordingly, the liquid reaction medium may comprise a mixture of water and one or more organic solvents.

To promote the desired reaction between the active hydrogen-containing organic compound and the halogenated olefin, it may be advantageous to conduct the contacting of the reactants under basic conditions. For example, one or more bases may be present in the reaction mixture; the base may be present in solubilized or insoluble form. The base may be a weak or a strong base, provided that it is not so strong that it leads to undesired side reactions of the halogenated organic compound which is the target product. Inorganic bases may be used, in particular alkali metal hydroxides (e.g., NaOH, KOH) and alkali metal salts of carbonic acid (e.g., potassium carbonate, sodium carbonate, cesium carbonate). Organic bases, in particular tertiary amines such as trialkylamines, pyridine and the like may also be employed. The use of basic ion exchange resins is also possible. The amount of base may be varied as may be desired depending upon the reactants and base used and other reaction conditions (temperature, solvent), but in one embodiment is approximately equimolar with respect to the moles of active hydrogen-containing organic compound used. More highly basic conditions (i.e., the use of a strong base or high pH) typically helps promote the formation of an alkenyl-containing product, which is believed to result from the elimination of hydrohalide from an initially formed haloalkyl-containing product.

Optionally, a phase transfer catalyst (PTC) may also or additionally be employed to promote the desired reaction between the halogenated olefin and the active hydrogen-containing organic compound. Any suitable phase transfer catalyst known in the field of organic chemistry may be employed such as, for example, ammonium compounds (e.g., quaternary ammonium compounds such as tetraalkylammonium halides or hydroxides), phosphonium compounds, crown ethers, cryptands (also referred to as cryptates), polyethylene glycols (PEG) and ethers thereof and other organo-based complexing agents. The phase transfer catalyst may be water soluble or organic soluble. Typically, if a phase transfer catalyst is used in combination with a base, the molar amount of phase transfer catalyst may be, for example, 0.1 to 5% of the molar amount of base.

Suitable illustrative quaternary ammonium salts include benzyldimethyltetradecylammonium chloride hydrate, benzylcetyldimethylammonium chloride hydrate, benzalkonium chloride, benzyltriethylammonium bromide, benzyltriethylammonium chloride, benzyltriethylammonium iodide, benzyltrimethylammonium chloride, benzyltributylammonium bromide, benzyltributylammonium chloride, benzyldodecyldimethylammonium chloride dihydrate, benzyltrimethylammonium bromide, benzyldodecyldimethylammonium bromide, bis(2-hydroxyethyl)dimethylammonium chloride, dodecyltrimethylammonium chloride, decyltrimethylammonium chloride, decyltrimethylammonium bromide, dodecyltrimethylammonium bromide, 4-dimethylamino-1-neopentylpyridinium chloride, dilauryldimethylammonium bromide, dimethyldioctadecylammonium bromide, diallyldimethylammonium chloride, dimethyldipalmitylammonium bromide, dimethyldimyristylammonium bromide, didecyldimethylammonium bromide, dimethyldioctylammonium bromide, dimethyldioctadecylammonium iodide, didodecyldimethylammonium chloride, ethyltrimethylammonium iodide, hexadecyltrimethylammonium bromide, hexadecyltrimethylammonium chloride, hexyltrimethylammonium bromide, triethylmethylammonium chloride, triethylphenylammonium chloride, trimethylphenylammonium bromide, trimethylphenylammonium chloride, trimethylphenylammonium tribromide, trimethylstearylammonium bromide, tetrabutylammonium bromide, tetrabutylammonium chloride, tetrabutylammonium iodide, tetraethylammonium bromide, tetraethylammonium chloride, tetraethylammonium iodide, tetramethylammonium bromide, tetramethylammonium chloride, tetramethylammonium iodide, tetrapropylammonium iodide, tetrapropylammonium bromide, trioctylmethylammonium, tetradecyltrimethylammonium bromide, trimethyltetradecylammonium chloride, tetrahexylammonium iodide, tetramethylammonium ccetate, tetra(decyl)ammonium bromide, tetra-n-octylammonium iodide, tetramethylammonium sulfate, tetrabutylammonium triiodide, methyltri-n-octylammonium chloride, tetraheptylammonium iodide, tetramethylammonium acetate, tetraamylammonium bromide, tetraamylammonium chloride, tetrahexylammonium bromide, tetraheptylammonium bromide, tetra-n-octylammonium bromide, tetrapropylammonium chloride, trimethyl[2-[(trimethylsilyl)methyl]benzyl]ammonium iodide, tetrabutylammonium acetate, trimethylnonylammonium bromide trimethylpropylammonium bromide, tributylmethylammonium, tetraethylammonium nitrate. Examples of suitable phosphonium salts include trans-2-butene-1,4-bis(triphenylphosphonium chloride), tributyldodecylphosphonium bromide, tributylhexadecylphosphonium bromide, tributyl-n-octylphosphonium bromide, tetrakis(hydroxymethyl)phosphonium chloride, tetraphenylphosphonium bromide, tetrakis(hydroxymethyl)phosphonium sulfate, tetrabutylphosphonium bromide, tetraphenylphosphonium chloride, tetraethylphosphonium bromide, tetrabutylphosphonium chloride, tetra-n-octylphosphonium bromide, tetraethylphosphonium hexafluorophosphate, tetraethylphosphonium, tetrafluoroborate, tetrabutylphosphonium tetrafluoroborate, and tetrabutylphosphonium hexafluorophosphate. Suitable crown ethers include, for example, 12-crown-4, 15-crown-5, 18-crown-6 and their complexes.

Reaction temperatures may vary, for example, from about 5° C. to about 200° C., e.g., from about 10° C. to about 150° C. or from about 20° C. to about 120° C. The pressure in the reactor is between ambient and 50 bar, preferably between ambient and 20 bar. The pressure may be the autogenous pressure of the solution, or an inert, for example nitrogen, may be added to increase the pressure. Typically, reaction times will range from about 0.5 hours to about 72 hours, e.g., from about 4 to about 12 hours.

The reactants may be combined all at once and then reacted. Alternatively, one or both of the active hydrogen-containing organic compound and the halogenated olefin may be added continuously or in portions or stages to the reaction mixture. If the active hydrogen-containing organic compound contains two or more active hydrogen-containing functional groups and it is desired to obtain a product in which at least one of the active hydrogen-containing functional groups remains unreacted, it may be preferred to add the halogenated olefin incrementally to the active hydrogen-containing organic compound while reacting the two reactants so as to favor the production of the desired product.

In certain embodiments of the invention, approximately stoichiometric amounts of the active hydrogen-containing organic compound and the halogenated olefin are employed, but in other embodiments a stoichiometric excess of one reactant may be used.

For instance, the active hydrogen-containing organic compound and the halogenated olefin may be reacted in a stoichiometric ratio of (moles active hydrogen-containing organic compound)/x:moles halogenated olefin, wherein x=number of active hydrogens per molecule of the active hydrogen-containing organic compound, of from about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1, or about 1:1.5 to about 1.5:1, or about 1:1.1 to about 1.1:1.

Where the active hydrogen-containing organic compound contains two or more active hydrogen-containing functional groups per molecule (e.g., where the active hydrogen-containing organic compound is an aliphatic polyalcohol) and it is desired to obtain a product, following reaction with a halogenated olefin, that contains one or more free (unreacted) active hydrogen-containing functional groups per molecule, it may be desirable to employ a stoichiometric excess of the active hydrogen-containing organic compound relative to the halogenated olefin so as to favor the production of such a product over a product where all the active hydrogen-containing functional groups have reacted with halogenated olefin. In such cases, the active hydrogen-containing organic compound and the halogenated olefin may be reacted in a stoichiometric ratio of (moles active hydrogen-containing organic compound)/x:moles halogenated olefin, wherein x=number of active hydrogens per molecule of the active hydrogen-containing organic compound, of from about 1x:1 to about 12x:1, about 1.5x:1 to about 10x:1 or about 2x:1 to about 8x:1.

### Purification

Once the reaction between the active hydrogen-containing organic compound and the halogenated olefin has been carried out for a desired period of time (e.g., to a predetermined degree of conversion of the starting materials), the reaction mixture obtained may be subjected to one or more further processing and/or purification steps in order to isolate the desired halogenated organic compound from the other components of the reaction mixture (e.g., solvent, unreacted starting materials, undesired byproducts, base, and so forth). Any of the purification techniques known in the organic chemistry field, or any combination of such techniques, may be employed, with the particular methods selected being influenced by various parameters such as the volatility, crystallizability, solubility, polarity, acidity/basicity and other such characteristics of the components of the reaction mixture. Suitable isolation/purification techniques include, but are not limited to, distillation (including fractional distillation), extraction, filtration, washing, neutralization, chromatographic separation, adsorption/absorption, treatment with ion exchange resin, crystallization, recrystallization, trituration, sublimation, precipitation, dialysis, membrane separation, filtration, centrifugation, decolorization, drying and the like and combinations thereof. By application of such techniques, the halogenated organic compound may be obtained in a purity (by weight) of at least 80%, at least 85%, at least 90%, at least 95%, at least 99% or even 100%.

In other embodiments of the invention the reaction mixture is not subjected to purification but instead is used as-is (or in only partially purified form) in one or more subsequent reaction steps wherein the halogenated organic compound present in the reaction mixture is converted to one or more other compounds.

### Further Reaction of the Halogenated Organic Compound Product

A halogenated organic compound produced in accordance with the present invention may be subjected to one or more further reactions to convert the halogenated organic compound into another compound of interest. Accordingly, halogenated organic compounds obtained by the processes described herein may function as synthetic intermediates. For example, one or more functional groups on the halogenated organic compound may be transformed or converted into other types of functional groups using reagents and conditions known in the field of organic chemistry.

In one embodiment, a carbon-carbon double bond in the halogenated organic compound may be reacted, e.g., hydrogenated to provide a saturated species, hydrohalogenated and/or halogenated to introduce additional halogen into the halogenated organic compound, oxidized, reacted with a diene to provide a Diels-Alder adduct, polymerized, or the like.

In another embodiment, a free active hydrogen-containing group in the halogenated organic compound (e.g., a hydroxyl group, a thiol group, a secondary amino group or a primary amino group) may be reacted with a compound containing active hydrogen-reactive functional group (e.g., an isocyanate group, a carboxylic acid group, an anhydride group, a carboxylic acid ester group, or an acyl halide group) which may optionally contain at least one other functional group (e.g., a (meth)acrylate group). This chemistry may be employed as a way of introducing one or more desired functional groups into the halogenated organic compound.

For example, a hydroxyl-functionalized halogenated organic compound may be reacted with a (meth)acrylic anhydride to provide a (meth)acrylate-functionalized halogenated organic compound. This synthetic route may be illustrated by the following general reaction scheme: HO—R—OH+CX¹X²═CX³X⁴→HO—R—O—CX¹X²—CX³X⁴H HO—R—O—CX¹X²—CX³X⁴+CH₂═CHR¹—C(═O)—O—C(═O)—CHR¹═CH₂→CH₂═CHR¹—C(═O)—O—R—O—CX¹X²—CX³X⁴H wherein R is an organic moiety, R¹ is H or CH₃, X¹, X², X³ and X⁴ are independently selected from the group consisting of hydrogen (H), chlorine (Cl), fluorine (F), bromine (Br), iodine (I) and halogenated and non-halogenated C1-C8 alkyl groups, subject to the provisos that one or more of X¹, X², X³ and X⁴ are halogens selected from the group consisting of chlorine (Cl), fluorine (F), bromine (Br) and iodine (I) and, when one of X¹, X², X³ or X⁴ is halogen and each of the other X¹, X², X³ and X⁴ substituents is a substituent other than halogen, the halogenated olefin contains at least one halogenated alkyl group. In a variation of this approach, one of the hydroxyl groups in the active hydrogen-containing compound starting material may be blocked (masked) while reacting the halogenated olefin with the active hydrogen-containing compound, with the blocking (masking group) being removed prior to reacting the intermediate halogenated organic compound with (meth)acrylic anhydride.

### Further Description of Processing Conditions

Further descriptions of exemplary, illustrative processes in accordance with the present invention are provided as follows:

### Product:

A halogenated olefin is reacted with an alcohol (e.g., an aliphatic alcohol, aliphatic polyalcohol or phenol) to produce a halogenated alkenyl ether. The reaction takes place in a heavy solvent and is catalyzed by a base.

### Process Overview:

A halogenated olefin and an alcohol are added to a solvent, e.g., DMSO, NMP, acetonitrile and the like, in the presence of a base, e.g. KOH, NaOH or the like. The reaction proceeds at temperatures between ambient and 200° C., typically between 20 and 100° C. for a period of between 1 and 24 hours, typically 2-4 hours. The pressure in the reactor is between ambient and 50 bar, preferably between ambient and 20 bar. The pressure may be the autogenous pressure of the solution, or an inert, for example nitrogen, may be added to increase the pressure. The product of the reaction is then purified. The purification methodology may be distillation, crystallization, and/or extraction, by themselves or together. The solvent may optionally be recycled and used for the next batch or remixed into the feed vessel in a continuous process.

### Process Description:

The process may be performed either continuously, semi-continuously or in batch mode. For the purpose of explanation a batch reactor will be discussed. However, a batch reactor is not required; it is only used to illustrate the process.

A typical feed mixture may be 1300 kg of the halogenated olefin, 1300 kg of an alcohol, 8580 kg of a solvent, and 800 kg of potassium hydroxide powder. For this example, DMSO is employed. In this example, excess alcohol is employed. Alternatively, equal molar amounts of halogenated olefin and alcohol or even excess halogenated olefin may be employed. The feed may be pre-mixed or added directly to the reactor.

The reaction may be performed in a stirred tank reactor. Coils inside the reactor and/or a jacket are used to cool or heat the reactor. The stirring is desirable. Other reactor configurations, e.g., a loop, tubular with internal or external heat exchange, may be employed. An optional static mixer may also be used. The reactor may be a pressurized reactor so as to facilitate conducting the reaction at a temperature above the boiling points of the reactants and (if present) solvent.

The reactor is heated to between ambient and 200° C., preferentially between 20 and 100° C. The reaction may be carried out at a pressure above atmospheric pressure, particularly where one or both of the reactants have significant volatility at the reaction temperature(s) (for example, where a reactant has a boiling point at atmospheric pressure below the reaction temperature to be utilized). The pressure in the reactor is between ambient and 50 bar, preferably between ambient and 20 bar. The pressure may be the autogenous pressure of the solution, or an inert, for example nitrogen, may be added to increase the pressure. The pressure within a reactor in which the halogenated olefin and the alcohol are being reacted may be generated by the reactants and any solvent which may be present or, additionally, by external pressure (e.g., pressurizing the head space above the liquid phase within a reactor using an inert gas such as nitrogen).

The reaction is typically carried out for between 0.25 and 24 hours. After the reaction, the contents may be pumped out of the reactor or kept in the reactor for processing. After the reaction, the contents of the reactor (i.e., the reaction mixture produced) may contain about 2300 kg of the halogenated alkenyl ether, 300 kg of the excess alcohol, 8580 kg of the solvent, and salts (KCl or KF, depending upon the particular halogenated olefin used).

Purification of the halogenated alkenyl ether may be accomplished by filtration, distillation or extraction or any other means known to the state of the art. Filtration is used to remove solids that are unreacted potassium hydroxide, potassium chloride or potassium fluoride depending on the particular halogenated olefin used. When employing distillation, the reactor is heated, optionally under vacuum or with pressure, to drive off the alcohol and halogenated alkenyl ether. Some of the solvent may also be distilled. This operation does not have to be performed in the same reactor, and could be done in a separate vessel or distillation column.

Continuing the example, 102,000 kg methylene chloride and 7020 kg water that is cooled to ambient temperature or close to 0° C., are added to the mixture in a continuous countercurrent Karr column. The aqueous stream product from the top of the column contains 7020 kg of water, 560 kg KCl, and about 7680 kg of the solvent DMSO. The organic phase is made up of 2300 kg halogenated alkenyl ether, 300 kg excess alcohol, and about 3900 kg DMSO in 93,500 kg CH₂Cl₂.

The organic phase is distilled to separate the extraction solvent from the heavier components. Distillation is also employed to remove the light alcohol for recycle, greater than 99.5% pure halogenated alkenyl ether and solvent. The distillation may be accomplished using any distillation technology known to the art including, but not limited to, trayed towers, packed towers, structured packed towers, divided wall towers, and the like.

The aqueous phase is subjected to distillation and crystallization, or precipitation, either together or in series. The stream is distilled to remove the water. At the same time, the salts are precipitated out in, e.g., a falling film crystallizer, or crystallized and filtered. In this way, the solvent may be recovered and recycled.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the composition or process. Additionally, in some embodiments, the invention can be construed as excluding any element or process step not specified herein.

Although the invention is illustrated and described herein with reference to specific embodiments, the invention is not intended to be limited to the details shown. Rather, various modifications may be made in the details within the scope and range of equivalents of the claims and without departing from the invention.

### EXAMPLES

### Example 1: Preparation of 4-chlorophenyl-3,3,3-trifluoropropenyl ether

A 100 ml four-neck (14/20) flask was set up in a heating mantle and placed on a magnetic stirrer. The flask was equipped with a thermowell that contained a thermocouple that was connected to a temperature controller and a dry-ice condenser with outlet connected to a nitrogen source. The reaction flask was charged with 4-chlorophenol (5.39 g/0.0419 mol), potassium carbonate 6.40 g/0.0463 mol) and DMSO (40.17 g/0.5129 mol). α,α,α-Trifluorotoluene (0.5196 g/0.0036 mol) was added as an internal standard. The reaction mixture was stirred while trans-(E)-1233zd (6.16 g/0.047 mol) was added subsurface through a septum over a 40 minute period. Following the addition of the 1233zd, the reaction mixture was heated to 70-90° C. for nine hours. Following the specified time, the reaction mixture was analyzed by NMR spectroscopy and the yield (based on internal standard) was 82% of the trans (E) isomer, 4-chlorophenyl-(E)-3,3,3-trifluoropropenyl ether, together with 4% of the cis (Z) isomer, 4-chlorophenyl-(Z)-3,3,3-trifluoropropenyl ether.

The reaction mixture was combined with 150 ml of water and 100 ml of methylene chloride and stirred for 15 minutes. The resulting mixture was placed in a separatory funnel where two immiscible layers formed after sitting for 15 minutes. The resulting layers were separated and the bottom organic layer was washed twice with 100 ml of water. The organic layer was separated and the solvent stripped at reduced pressure to isolate the product. The amount of product isolated was 6.63 g. NMR analysis revealed a product with an isomer distribution of 94% trans-(E)-isomer and 6% cis-(Z)-isomer. There were identified about 3% impurities and, thus, the isolated yield was about 6.43 g, representing a 69% isolated yield (based on starting phenol).

### Characterization Data: 4-chlorophenyl-3,3,3-trifluoropropenyl ether

¹H NMR (CDCl₃): δ 5.38 ppm (doq, 1H, ³J_{H-H}=13 Hz, ³J_{H-F}=7 Hz); δ 7.21 ppm (doq, 1H, ³J_{H-H}=13 Hz, ⁴J_{H-F}=2 Hz); δ 6.90-7.40 ppm (m, 4H).

¹⁹F NMR (CDCl₃): trans-isomer δ −60.60 (dod, 3F, ³J_{F-H}=7 Hz, ⁴J_{F-H}=2 Hz).

cis-isomer δ −58.13 (d, 3F, ³J=9 Hz). nD₂₀=1.4842.

Following the procedure described in Example 1, other derivatives were prepared from 1233zd in a similar fashion and the results are summarized in Table 1:

TABLE 1
Summary of Results from Examples 1-10

| Example | Phenol | Product (g) | Isolated Yield (%) |
|---|---|---|---|
| 1 | 4-Cl | 6.43 | 69 |
| 2 | 4-OH | 5.04 | 61 |
| 3 | 4-F | 6.99 | 81 |
| 4 | 4-CH₃ | 8.28 | 83 |
| 5 | 3-CN | 7.65 | 84 |
| 6 | 2-F | 6.65 | 73 |
| 7 | 3-NO₂ | 8.11 | 92 |
| 8 | 2,4-dichloro | 7.46 | 83 |
| 9 | 2-chloro-4-fluoro- | 7.22 | 77 |
| 10 | 4-SO₃_{^{−}} | 4.65 | 49 |
| | Na⁺ | | |

### Characterization Data, Examples 2-10

### Example 2: 1,4-bis(3,3,3-trifluoropropenyloxy)benzene

¹H NMR (CDCl₃): δ 5.35 ppm (doq, 1H, ³J_{H-H}=12 Hz, ³J_{H-F}=7 Hz); δ 7.22 ppm (doq, 1H, ³J_{H-H}=12 Hz, ⁴J_{H-F}=2 Hz); δ 7.06 ppm (s, 4H).

¹⁹F NMR (CDCl₃): trans-isomer δ −60.73 (dod, 3F, ³J_{F-H}=7 Hz, ⁴J_{F-H}=2 Hz).

cis-isomer δ −58.29 (d, 3F, ³J=9 Hz). nD₂₀=1.4516.

### Example 3: 4-fluorophenyl-3,3,3-trifluoropropenyl ether

¹H NMR (CDCl₃): δ 5.32 ppm (doq, 1H, ³J_{H-H}=12 Hz, ³J_{H-F}=7 Hz); δ 7.21 ppm (doq, 1H, ³J_{H-H}=12 Hz, ⁴J_{H-F}=2 Hz); δ 7.04 ppm (m, 4H).

¹⁹F NMR (CDCl₃): trans-isomer δ −60.65 (dod, 3F, ³J_{F-H}=7 Hz, ⁴J_{F-H}=3 Hz).

cis-isomer δ −58.23 (d, 3F, ³J=8 Hz). nD₂₀=1.4434.

### Example 4: 4-methylphenyl-3,3,3-trifluoropropenyl ether

¹H NMR (CDCl₃): δ 2.35 (s, 3H); δ 5.31 ppm (doq, 1H, ³J_{H-H}=12 Hz, ³J_{H-F}=7 Hz); δ 7.24 ppm (doq, 1H, ³J_{H-H}=12 Hz, ⁴J_{H-F}=2 Hz); δ 7.17 ppm (m, 4H).

¹⁹F NMR (CDCl₃): trans-isomer δ −60.51 (d, 3F, ³J_{F-H}=6 Hz).

cis-isomer δ −58.12 (d, 3F, ³J=9 Hz). nD₂₀=1.4624.

### Example 5: 3-cyanophenyl-3,3,3-trifluoropropenyl ether

¹H NMR (CDCl₃): δ 5.51 ppm (doq, 1H, ³J_{H-H}=12 Hz, ³J_{H-F}=7 Hz); δ 7.27 ppm (doq, 1H, ³J_{H-H}=12 Hz, ⁴J_{H-F}=2 Hz); δ 7.30-7.57 ppm (m, 4H).

¹⁹F NMR (CDCl₃): trans-isomer δ −60.95 (d, 3F, ³J_{F-H}=5 Hz).

cis-isomer δ −58.35 (d, 3F, ³J=8 Hz). nD₂₀=1.4915.

### Example 6: 2-fluorophenyl-3,3,3-trifluoropropenyl ether

¹H NMR (CDCl₃): δ 5.30 ppm (doq, 1H, ³J_{H-H}=12 Hz, ³J_{H-F}=7 Hz); δ 7.00-7.30 ppm (m, 4H).

¹⁹F NMR (CDCl₃): trans-isomer δ −60.66 (d, 3F, ³J_{F-H}=6 Hz).

cis-isomer δ −58.16 (d, 3F, ³J=8 Hz); δ −131.80 (m, 1F). nD₂₀=1.4421.

### Example 7: 3-nitrophenyl-3,3,3-trifluoropropenyl ether

¹H NMR (CDCl₃): δ 5.53 ppm (doq, 1H, ³J_{H-H}=12 Hz, ³J_{H-F}=7 Hz); δ 7.28 ppm (doq, 1H, ³J_{H-H}=12 Hz, ⁴J_{H-F}=2 Hz); δ 7.35-8.10 ppm (m, 4H).

¹⁹F NMR (CDCl₃): trans-isomer δ −61.28 (d, 3F, ³J_{F-H}=7 Hz).

cis-isomer δ −58.71 (d, 3F, ³J=8 Hz). nD₂₀=1.4977.

### Example 8: 2,4-dichlorophenyl-3,3,3-trifluoropropenyl ether

¹H NMR (CDCl₃): δ 5.31 ppm (doq, 1H, ³J_{H-H}=12 Hz, ³J_{H-F}=7 Hz); δ 7.17 ppm (doq, 1H, ³J_{H-H}=12 Hz, ⁴J_{H-F}=2 Hz); δ 6.92-7.48 ppm (m, 3H).

¹⁹F NMR (CDCl₃): trans-isomer δ −61.07 (dod, 3F, ³J_{F-H}=6 Hz, ⁴J_{F-H}=2 Hz). cis-isomer □−58.61 (d, 3F, ³J=8 Hz). nD₂₀=1.4979.

HRMS [M′]⁺=255.9669 m/z (observed); 255.9670 m/z (calc).

### Example 9: 2-chloro-4-fluorophenyl-3,3,3-trifluoropropenyl ether

¹H NMR (CDCl₃): δ 5.23 ppm (doq, 1H, ³J_{H-H}=12 Hz, ³J_{H-F}=7 Hz); δ 7.16 ppm (doq, 1H, ³J_{H-H}=12 Hz, ⁴J_{H-F}=2 Hz); δ 6.96-7.26 ppm (m, 3H).

¹⁹F NMR (CDCl₃): trans-isomer δ −60.74 (d, 3F, ³J_{F-H}=7 Hz, ⁴J_{F-H}=2 Hz). cis-isomer δ −58.33 (d, 3F, ³J=8 Hz); Aromatic-F δ −114.92 (m, 1F). nD₂₀=1.4633.

HRMS [M′]⁻=239.9960 m/z (observed); 239.9960 m/z (calc).

### Example 10: 4-(3,3,3-trifluoropropenyl)phenyl sulfate, sodium salt

¹H NMR (CDCl₃): trans δ 5.79 ppm (doq, 1H, ³J_{H-H}=12 Hz, ³J_{H-F}=7 Hz); δ 7.6 not fully resolved; δ 7.06-7.64 ppm (m, 4H). cis δ 5.32 ppm (doq, 1H, ³J_{H-F}=8 Hz, ³J_{H-H}=7 Hz); δ 7.21 ppm (d, 1H, ³J_{H-H}=7 Hz).

¹⁹F NMR (CDCl₃): trans-isomer δ −57.69 (d, 3F, ³J_{F-H}=7 Hz, ⁴J_{F-H}=2 Hz). cis-isomer δ −55.45 (d, 3F, ³J=8 Hz).

### Example 11: Preparation of 4-fluorophenyl-3,3,3-trifluoroprop-2-enyl ether

Following a similar procedure to that described in Example 1, with the exception that 1233xf was used in place of 1233zd, 5.23 g (46.7 mmol) of 4-fluorophenol together with 7.54 g (54.6 mmol) potassium carbonate in 45.05 g (520.5 mmol) DMSO was reacted with 9.90 g (75.9 mmol) 1233xf at 70-90° C. over 8 hours. After aqueous work-up similar to that described in Example 1, 8.63 g of 97% pure product was obtained. Analysis by NMR spectroscopy confirmed the identity of the product. The isolated yield of the titled product was 8.37 g=87.0%.

### Characterization Data: 4-fluorophenyl-3,3,3-trifluoroprop-2-enyl ether

¹H NMR (CDCl₃): δ 5.00 ppm (doq, 1H, ³J_{H-H}=8 Hz, ³J_{H-F}=8 Hz); δ 6.66 ppm (d, 1H, ³J_{H-H}=8 Hz). δ 7.02-7.25 ppm (m, 4H).

¹⁹F NMR (CDCl₃): δ −58.07 (d, 3F, ³J_{F-H}=8 Hz). nD₂₀=1.4983.

Following the procedure described in Example 11, other derivatives were prepared from 1233xf in a similar fashion. The results obtained are summarized in Table 2:

TABLE 2
Summary of Results from Examples 11-20.

| Example | Phenol | Product (g) | Isolated Yield (%) |
|---|---|---|---|
| 11 | 4-F | 8.37 | 87 |
| 12 | 3-NO₂ | 5.00 | 61 |
| 13 | 2-F | 4.62 | 55 |
| 14 | 4-CH₃ | 7.69 | 68 |
| 15 | 4-Cl | 7.97 | 85 |
| 16 | 3-CN | 7.60 | 81 |
| 17 | 4-OH | 5.64 | 70 |
| 18 | 2,4-dichloro | 5.37 | 63 |
| 19 | 2-chloro-4-fluoro- | 4.57 | 49 |
| 20 | 4-SO₃_{^{−}}Na⁺ | 7.44 | 79 |

### Characterization Data, Examples 12-20

### Example 12: 3-nitrophenyl-3,3,3-trifluoroprop-2-enyl ether

¹H NMR (CDCl₃): δ 5.21 ppm (doq, 1H, ²J_{H-H}=7 Hz, ⁴J_{H-F}=8 Hz); δ 6.82 ppm (d, 1H, ²J_{H-H}=7 Hz); δ 7.42 ppm (m, 1H); δ 7.58 ppm (t, 1H, J_{H}-H=8 Hz); δ 7.91 ppm (t, 1H, J_{H-H}=2 Hz); δ 8.05 ppm (m, 1H).

¹⁹F NMR (CDCl₃): δ −58.42 (d, 3F, ⁴J_{F-H}=8 Hz). nD₂₀=1.5123.

HRMS [M−H]⁻=232.0233 m/z (observed); 232.0227 m/z (calc).

### Example 13: 2-fluorophenyl-3,3,3-trifluoroprop-2-enyl ether

¹H NMR (CDCl₃): δ 5.02 ppm (doq, 1H, ²J_{H-H}=7 Hz, ⁴J_{H-F}=8 Hz); δ 6.65 ppm (dod, 1H, ²J_{H-H}=7 Hz, ⁴J_{H-F}=2 Hz); δ 7.05 to 7.25 ppm (m, 4H).

¹⁹F NMR (CDCl₃): δ −58.42 (d, 3F, ⁴J_{F-H}=8 Hz). Aromatic-F δ −133.40 (m, 1F). nD₂₀=1.4505.

HRMS [M−H]⁻=205.0280 m/z (observed); 205.0282 m/z (calc).

### Example 14: 4-methylphenyl-3,3,3-trifluoroprop-2-enyl ether

¹H NMR (CDCl₃): δ 4.93 ppm (doq, 1H, ²J_{H-H}=7 Hz, ⁴J_{H-F}=8 Hz); δ 6.67 ppm (d, 1H, ²J_{H-H}=7 Hz); δ 2.30 ppm (s, 3H); δ 6.88 to 7.13 ppm (m, 4H).

¹⁹F NMR (CDCl₃): δ −58.11 (d, 3F, ⁴J_{F-H}=8 Hz). nD₂₀=1.4721.

HRMS [M′]⁺=202.0602 m/z (observed); 206.0600 m/z (calc).

### Example 15: 4-chlorophenyl-3,3,3-trifluoroprop-2-enyl ether

¹H NMR (CDCl₃): δ 5.03 ppm (doq, 1H, ²J_{H-H}=8 Hz, ⁴J_{H-F}=8 Hz); δ 6.67 ppm (d, 1H, ²J_{H-H}=8 Hz); δ 6.95 to 7.35 ppm (m, 4H).

¹⁹F NMR (CDCl₃): δ −58.30 (d, 3F, ⁴J=8 Hz). nD₂₀=1.4896.

HRMS [M′]⁺=222.0057 m/z (observed); 222.0054 m/z (calc).

### Example 16: 3-cyanophenyl-3,3,3-trifluoroprop-2-enyl ether

¹H NMR (CDCl₃): δ 5.17 ppm (doq, 1H, ²J_{H-H}=7 Hz, ⁴J_{H-F}=8 Hz); δ 6.73 ppm (d, 1H, ²J_{H-H}=7 Hz); δ 7.29 to 7.54 ppm (m, 4H).

¹⁹F NMR (CDCl₃): δ −58.50 (d, 3F, ⁴J=8 Hz). nD₂₀=1.4963.

HRMS [M−H]⁻=212.0334 m/z (observed); 212.0329 m/z (calc).

### Example 17: 1,4-bis(3,3,3-trifluoroprop-2-enyl)phenyl ether

¹H NMR (CDCl₃): δ 5.03 ppm (doq, 1H, ²J_{H-H}=7 Hz, ⁴J_{H-F}=8 Hz); δ 6.68 ppm (d, 1H, ²J_{H-H}=7 Hz); δ 7.07 ppm (s, 4H).

¹⁹F NMR (CDCl₃): δ −58.28 (d, 3F, ⁴J=8 Hz); nD₂₀=1.4434.

HRMS [M′]⁺=298.0430 m/z (observed); 298.0423 m/z (calc).

### Example 18: 2,4-dichlorophenyl-3,3,3-trifluoroprop-2-enyl ether

¹H NMR (CDCl₃): δ 5.09 ppm (doq, 1H, ²J_{H-H}=7 Hz, ⁴J_{H-F}=8 Hz); δ 6.57 ppm (d, 1H, ²J_{H-H}=7 Hz); δ 7.00-7.50 ppm (m, 3H). ¹⁹F NMR (CDCl₃): δ −58.41 (d, 3F, ⁴J_{F-H}=8 Hz). nD₂₀=1.5124.

HRMS [M′]⁺=255.9669 m/z (observed); 255.9670 m/z (calc).

### Example 19: 2-chloro-4-fluorophenyl-3,3,3-trifluoroprop-2-enyl ether

¹H NMR (CDCl₃): δ 5.05 ppm (doq, 1H, ²J_{H-H}=7 Hz, ⁴J_{H-F}=8 Hz); δ 6.54 ppm (d, 1H, ²J_{H-H}=7 Hz); δ 6.85-7.25 ppm (m, 3H).

¹⁹F NMR (CDCl₃): δ −58.27 (d, 3F, ⁴J_{F-H}=8 Hz); Aromatic-F δ −115.6 (m, 1F). nD₂₀=1.4705.

### Example 20: 4-(3,3,3-trifluoroprop-2-enyl)phenyl sulfate, sodium salt

¹H NMR (CDCl₃): trans δ 5.38 ppm (doq, 1H, ⁴J_{H-F}=9 Hz, ²J_{H-H}=7 Hz); δ 7.27 (d, 1H, ²J_{H-H}=7); δ 7.08-7.65 ppm (m, 4H).

¹⁹F NMR (CDCl₃): δ −55.45 (d, 3F, ⁴J_{F-H}=9 Hz).

### Example 21: Preparation of 1-(3,3,3-trifluoroprop-1-enyl)imidazole

Following a similar procedure to that described in Example 1, with the exception that imidazole, 3.00 g (44.1 mmol) was used in place of 4-chlorophenol together with 6.49 g (47.0 mmol) potassium carbonate in 45.05 g (512.9 mmol) DMSO, and was reacted with 10.89 g (83.4 mmol) 1233zd at 140° C. over 17 hours. After aqueous work up similar to that described in Example 1 and sublimation, 1.19 g of a 99% pure oily solid was obtained. Analysis by NMR spectroscopy confirmed the identity of the product. The isolated yield of the titled product was 1.18 g=17.0%.

### Characterization Data: 1-(3,3,3-trifluoroprop-1-enyl)imidazole

¹H NMR (CDCl₃): trans-isomer δ 5.89 ppm (doq, 1H, ³J_{H-H}=14 Hz, ³J_{H-F}=6 Hz); δ 7.43 ppm (doq, 1H, ³J_{H-H}=14 Hz, ⁴J_{H-F}=2 Hz).

cis-isomer δ 5.42 ppm (doq, 1H, ³J_{H-H}=11 Hz, ³J_{H-F}=9 Hz); δ 6.96 ppm (d, 1H, ³J_{H-H}=11 Hz). Imidazole ring. δ 7.16 ppm (d, 1H, J=1 Hz); 7.19 ppm (t, 1H, J=1 Hz); δ 7.71 ppm (s, 1H).

¹⁹F NMR (CDCl₃): trans-isomer δ −62.58 (dod, 3F, ³J_{F-H}=6 Hz, ⁴J_{F-H}=2 Hz). cis-isomer δ −57.95 (d, 3F, ³J=8 Hz).

HRMS [M+H]⁺=163.0472 m/z (observed); 163.0478 m/z (calc).

### Example 22: Preparation of 1-(3,3,3-trifluoroprop-1-enyl)imidazole

Following a similar procedure to that described in Example 20, with the exception that the K⁺ imidazolium salt was pre-formed and the resulting salt then treated with 1233zd: 6.88 g (101.1 mmol) imidazole was treated with KOH, 6.89 g (122.8 mmol), used in place of potassium carbonate, and THF 102.35 g (1.4194 mol) was used in place of DMSO. 16.78 g (111.2 mmol) of 1233zd was reacted with this mixture at 60° C. over 53 hours. After aqueous work up similar to that described in Example 19, the crude product was analyzed by NMR spectroscopy to confirm the identity of the product. The isolated yield of the titled product was 3.47 g=21.0%.

### Example 23: Attempted Preparation of 1-(3,3,3-trifluoroprop-2-enyl)imidazole

Following a similar procedure to that described in Example 1, with the exception that imidazole, 3.00 g (44.1 mmol) was used in place of 4-chlorophenol, together with 6.50 g (47.1 mmol) potassium carbonate in 46.06 g (589.5 mmol) DMSO that was reacted with 9.40 g (72.0 mmol) 1233xf at 140° C. over 24 hours. After aqueous work up similar to that described in Example 1 and distillation under 1 Torr vacuum at 120 to 140° C., 0.75 g of a 98% pure product was obtained. Analysis by NMR spectroscopy confirmed the identity of the product to be the same two isomers as that observed in Example 20 (using 1233zd as the source of the trifluoropropyl moiety). The isolated yield of the titled product was 0.74 g=10.0%.

### Characterization Data

### 1-(3,3,3-trifluoroprop-1-enyl)imidazole

¹H NMR (CDCl₃): trans-isomer δ 5.88 ppm (doq, 1H, ²J_{H-H}=14 Hz, ⁴J_{H-F}=6 Hz); δ 7.42 ppm (doq, 1H, ²J_{H-H}=14 Hz, ⁴J_{H-F}=2 Hz).

Imidazole ring. δ 7.16 ppm (d, 1H, J=1 Hz); δ 7.19 ppm (t, 1H, J=1 Hz); δ 7.71 ppm (s, 1H). product isomer δ 5.42 ppm (doq, 1H, ²J_{H-H}=10 Hz, ⁴J_{H-F}=9 Hz); δ 6.95 ppm (d, 1H, ³J_{H-H}⁼11 Hz). Imidazole ring. δ 7.13 ppm (d, 1H, J=1 Hz); δ 7.26 ppm (t, 1H, J=1 Hz); δ 7.69 ppm (s, 1H).

¹⁹F NMR (CDCl₃): trans-isomer δ −62.51 (dod, 3F, ⁴J_{F-H}=6 Hz, ⁴J_{F-H}=2 Hz). cis-isomer δ −57.88 (dod, 3F, ⁴J_{F-H}=9 Hz, ⁴J_{F-H}=1 Hz).

HRMS [M+H]⁺=163.0473 m/z (observed); 163.0478 m/z (calc).

### Example 24: Preparation of 1-(3,3,3-trifluoroprop-1-enyl)pyrazole

Following a similar procedure to that described in Example 1, with the exception that pyrazole, 3.02 g (44.4 mmol) was used in place of 4-chlorophenol, together with 6.56 g (47.5 mmol) potassium carbonate in 45.72 g (585.2 mmol) DMSO that was reacted with 6.33 g (48.5 mmol) 1233zd at 140° C. over 24 hours. After aqueous work up similar to that described in Example 1 and distillation under 1 Torr vacuum at 120 to 140° C., 0.79 g of a 98% pure product was obtained. Analysis by NMR spectroscopy confirmed the identity of the target product as two isomers. The isolated yield of the titled product was 0.77 g=11.0%.

### Characterization Data: 1-(3,3,3-trifluoroprop-1-enyl)pyrazole

¹H NMR (CDCl₃): trans-isomer 6.26 ppm (doq, 1H, ³J_{H-H}=14 Hz, ³J_{H-F}=6 Hz); δ 7.48 ppm (doq, 1H, ³J_{H-H}=14 Hz, ⁴J_{H-F}=2 Hz).

Pyrazole ring. δ 7.69 ppm (s, 1H); δ 7.59 ppm (d, 1H, J=3 Hz); δ 6.43 ppm (t, 1H, J=2 Hz).

Product isomer δ 5.29 ppm (doq, 1H, ³J_{H-H}=10 Hz, ⁴J_{H-F}=9 Hz); δ 7.22 ppm (d, 1H, ³J_{H-H}=10 Hz).

¹⁹F NMR (CDCl₃): trans-isomer δ −62.25 (dod, 3F, ³J_{F-H}=7 Hz, ⁴J_{F-H}=2 Hz). cis-isomer δ −57.56 (d, 3F, ³J_{F-H}=9 Hz).

HRMS [M+H]⁺=163.0474 m/z (observed); 163.0478 m/z (calc).

### Example 25: Attempted Preparation of 1-(3,3,3-trifluoroprop-2-enyl)pyrazole

Following a similar procedure to that described in Example 1, with the exception that pyrazole, 3.23 g (47.4 mmol) was used in place of 4-chlorophenol, together with 7.14 g (51.7 mmol) potassium carbonate in 46.20 g (591.3 mmol) DMSO that was reacted with 8.10 g (62.1 mmol) 1233xf at 140° C. over 19 hours. After aqueous work up similar to that described in Example 1 and distillation under 1 Torr vacuum at 120 to 140° C., 0.49 g of a 99% pure product was obtained. Analysis by NMR spectroscopy confirmed the identity of the product to be the same two isomers as that observed in Example 23 (using 1233zd as the source of the trifluoropropyl moiety). The isolated yield of the titled product was 0.49 g=6.0%.

### Characterization Data: 1-(3,3,3-trifluoroprop-1-enyl)pyrazole

¹H NMR (CDCl₃): trans-isomer δ 6.25 ppm (doq, 1H, ²J_{H-H}=14 Hz, ⁴J_{H-F}=7 Hz); δ 7.48 ppm (doq, 1H, ²J_{H-H}=14 Hz, ⁴J_{H-F}=2 Hz).

Pyrazole ring. δ 7.69 ppm (s, 1H); δ 7.59 ppm (d, 1H, J=2 Hz); δ 6.43 ppm (t, 1H, J=2 Hz).

Product isomer δ 5.29 ppm (doq, 1H, ³J_{H-H}=11 Hz, ⁴J_{H-F}=9 Hz); δ 7.21 ppm (d, 1H, ³J_{H-H}=11 Hz).

¹⁹F NMR (CDCl₃): trans-isomer δ −62.21 (dod, 3F, ⁴J_{F-H}=7 Hz, ⁴J_{F-H}=2 Hz). cis-isomer δ −57.53 (dod, 3F, ⁴J_{F-H}=9 Hz, ⁴J_{F-H}=1 Hz).

HRMS [M+H]⁺=163.0472 m/z (observed); 163.0478 m/z (calc).

### Example 26: Reaction of 1233zd with Hydroxy-functionalized Tertiary Amine

Jeffcat© Z110 [HOCH₂CH₂N(CH₃)CH₂CH₂N(CH₃)₂] and 1233zd were combined and aged for two weeks at 50° C. in the presence of potassium hydroxide (KOH). Analysis by ¹H NMR confirmed partial reaction of the starting materials, in accordance with the following scheme:

HOCH₂CH₂N(CH₃)CH₂CH₂N(CH₃)₂+CF₃CH═CHCl→CF₃CH═CHOCH₂CH₂N(CH₃)CH₂CH₂N(CH₃)₂

### Example 27: Synthesis of 2,2-Dimethyl-4-(2-chloro-1,1-difluoroethoxymethyl)-1,3-dioxolane, Using Excess Solketal for Solvent

A 100 ml four-neck (14/20) flask was set up in a heating mantle and placed on a magnetic stirrer. The flask was equipped with a thermowell that contained a thermocouple that was connected to a temperature controller and a dry-ice condenser with outlet connected to a nitrogen source. The reaction flask was charged with 2,2-dimethyl-1,3-dioxolane-4-methanol (Solketal) (30.94 g/0.2341 mol), tetrabutylammonium bromide (0.15 g/0.0005 mol) and potassium hydroxide (3.36 g/0.0599 mol) dissolved in water (6.72 g/0.3733 mol). α,α,α-trifluorotoluene (0.4985 g/0.0034 mol) was added as an internal standard. The reaction mixture was stirred while 1-chloro-2,2-difluoroethylene (HCFC1122) (5.44 g/0.0552 mol) was added subsurface through a septum over a 10 minute period. The temperature ranged from 17° C. to 33° C. at the end of addition. Following the addition of HCFC1122, the reaction mixture was stirred at ambient temperature for two hours.

The reaction mixture was combined with 150 ml of water and 100 ml of methylene chloride and stirred for 15 minutes. The resulting mixture was placed in a separatory funnel where two immiscible layers formed after sitting for 15 minutes. The resulting layers were separated and the bottom organic layer was washed twice with 100 ml of water. The organic layer was separated and the solvent stripped at reduced pressure to isolate the product. The amount of product isolated was 10.05 g. The major product was 2,2-dimethyl-4-(2-chloro-1,1-difluoroethoxymethyl)-1,3-dioxolane. The purity was 42 wt % and the yield was 33% based on FNMR internal standard analysis. The ketal blocking group could be removed from this product to yield a dihydroxy-functionalized compound bearing a —O—CClH—CF₂H group.

¹⁹F NMR (CDCl₃): δ −79.64 (F_{A}), −79.89 (F_{B}) ppm, q of t, ²J_{Fa-Fb}=−140 Hz, ³J_{H-F}=9 Hz

The chemical shifts of F_{A} and F_{B} were calculated from the AB type quartet.

### Example 28: Synthesis of 2,2-Dimethyl-4-(2-chloro-1,1-difluoroethoxymethyl)-1,3-dioxolane, Using DMSO for Solvent

A 100 ml four-neck (14/20) flask was set up in a heating mantle and placed on a magnetic stirrer. The flask was equipped with a thermowell that contained a thermocouple that was connected to a temperature controller and a dry-ice condenser with outlet connected to a nitrogen source. The reaction flask was charged with Solketal (6.62 g/0.0500 mol), DMSO (54.22 g/0.6940 mol), tetrabutylammonium bromide (0.15 g/0.0005 mol) and potassium hydroxide (2.86 g/0.0509 mol) dissolved in water (5.72 g/0.3178 mol). The reaction mixture was stirred while 1-chloro-2,2-difluoroethylene (HCFC1122) (5.44 g/0.0552 mol) was added subsurface through a septum over a 10 minute period. The temperature increased from 22° C. to 45° C. at the end of addition. Following the addition of HCFC1122, the reaction mixture was stirred at ambient temperature for sixteen hours.

The reaction mixture was combined with 150 ml of water and 100 ml of methylene chloride and stirred for 15 minutes. The resulting mixture was placed in a separatory funnel where two immiscible layers formed after sitting for 15 minutes. The resulting layers were separated and the bottom organic layer was washed twice with 100 ml of water. The organic layer was separated and the solvent stripped at reduced pressure to isolate the product. The amount of product isolated was 9.93 g. The major product was 2,2-dimethyl-4-(2-chloro-1,1-difluoroethoxymethyl)-1,3-dioxolane. The purity was 52 wt % and the yield was 45% based on FNMR internal standard analysis.

¹⁹F NMR (CDCl₃): δ −79.64 (F_{A}), −79.89 (F_{B}) ppm, q of t, ²J_{Fa-Fb}=−140 Hz, ³J_{H-F}=9 Hz

The chemical shifts of F_{A} and F_{B} were calculated from the AB type quartet.

### Example 29 Synthesis of 2,2-Dimethyl-4-[(1-fluoroethenyloxy)methyl]-1,3-dioxolane Using DMSO for Solvent

A 100 ml four-neck (14/20) flask was set up in a heating mantle and placed on a magnetic stirrer. The flask was equipped with a thermowell that contained a thermocouple that was connected to a temperature controller and a dry-ice condenser with outlet connected to a nitrogen source. The reaction flask was charged with Solketal (5.60 g/0.0424 mol), DMSO (31.10 g/0.3989 mol), and potassium hydroxide (2.67 g/0.0476 mol). The reaction mixture was stirred while 1-Chloro-1-fluoroethylene (HCFC1131a) (5.44 g/0.0552 mol) was added subsurface through a septum over a 5 minutes period. The temperature increased from 23° C. to 41° C. at the end of addition. Following the addition of HCFC1131a, the reaction mixture was stirred at ambient temperature for 48 hours.

The reaction mixture was combined with 150 ml of water and 100 ml of methylene chloride and stirred for 15 minutes. The resulting mixture was placed in a separatory funnel where two immiscible layers formed after sitting for 15 minutes. The resulting layers were separated and the bottom organic layer was washed twice with 100 ml of water. The organic layer was separated and the solvent stripped at reduced pressure to isolate the product. The amount of product isolated was 5.90 g. The major product was 2,2-Dimethyl-4-[(1-fluoroethenyloxy]methyl)-1,3-dioxolane. The purity was 72 wt % and the yield was 52% based on FNMR internal standard analysis.

¹⁹F NMR (CDCl₃): δ −80.62 ppm (d of d, ³J_{F-H}=41.3 Hz (trans), 6.5 Hz cis)

¹HNMR (CDCl₃): δ 3.24-3.40 (d of d, 1H-trans, ³J_{H-F}=41.3, ²J_{H-H}=4.4); δ 3.60-3.65, (d of d,

1H-cis, ³J_{H-F}=6.5, ²J_{H-H}=4.4); δ 3.74-3.64 (m, 3H), δ 4.06-4.12 (m, 1H), δ 4.32-4.40 (m, 1H)

### Example 30 Synthesis of 2,2-Dimethyl-4-[(1-fluoroethenyloxy]methyl)]-1,3-dioxolane Using DMSO for Solvent

A 100 ml four-neck (14/20) flask was set up in a heating mantle and placed on a magnetic stirrer. The flask was equipped with a thermowell that contained a thermocouple that was connected to a temperature controller and a dry-ice condenser with outlet connected to a nitrogen source. The reaction flask was charged with Solketal (5.69 g/0.0431 mol), DMSO (31.48 g/0.4029 mol), and potassium hydroxide (3.20 g/0.0520 mol). The reaction mixture was stirred while 1-Chloro-1-fluoroethylene (HCFC1131a) (4.81 g/0.0598 mol) was added subsurface through a septum over a 8 minutes period. The temperature increased from 23° C. to 55° C. at the end of addition. Following the addition of HCFC1131a, the reaction mixture was stirred at ambient temperature for 16 hours.

Hexane (50 ml) was added and the reaction mixture was heated with stirring to 50° C. for one hour. The reaction mixture was cooled to ambient temperature (22° C.) and the stirring was stopped. The layers were allowed to settle for 15 minutes. The top hexane layer was removed by siphoning with a syringe. A second 50 ml of hexane was charged to the reaction flask and the mixture was stirred for 15 minutes at ambient temperature. The stirring was stopped and the layers were allowed to settle for 15 minutes. The top hexane layer was removed by siphoning with a syringe. The two hexane extractions were combined and the solvent was stripped at reduced pressure to isolate the product. The amount of product isolated was 4.05 g. The major product was 2,2-Dimethyl-4-(2-chloro-1,1-difluoroethoxymethyl)-1,3-dioxolane. The purity was 75 wt % and the yield was 41% based on FNMR internal standard analysis. The remaining reaction mixture was combined with 150 ml of water and 100 ml of methylene chloride and stirred for 15 minutes. The resulting mixture was placed in a separatory funnel where two immiscible layers formed after sitting for 15 minutes. The resulting layers were separated and the bottom organic layer was washed twice with 100 ml of water. The organic layer was separated and the solvent stripped at reduced pressure to isolate the product. The amount of product isolated was 3.40 g. The major product was 2,2-Dimethyl-4-[(1-fluoroethenyloxy)methyl)]-1,3-dioxolane. The purity was 57 wt % and the yield was 26% based on FNMR internal standard analysis.

¹⁹F NMR (CDCl₃): δ −80.62 ppm (d of d, ³J_{F-H}=41.3 Hz (trans), 6.5 Hz cis)

¹HNMR (CDCl₃): δ 3.24-3.40 (d of d, 1H-trans, ³J_{H-F}=41.3, ²J_{H-H}=4.4); δ 3.60-3.65, (d of d, 1H-cis, ³J_{H-F}=6.5, ²J_{H-H}=4.4); δ 3.74-3.64 (m, 3H), δ 4.06-4.12 (m, 1H), δ 4.32-4.40 (m, 1H)

### Example 31 Reaction of 1,1,2-Trifluoro-2-chloroethylene (CTFE) with 2-Hydroxyethylmethacrylate (HEMA) in 30% Acetone and 70% DMSO Solvent

A 1 L four-neck (14/20) flask with overhead stirring and equipped with a digital thermometer and a dry-ice condenser with outlet connected to a nitrogen source. A pre-puncture septum was placed on the remaining neck. The reaction flask was charged with 2-Hydroxyethylmethacrylate (80.22 g/0.6160 mol), DMSO (374.66 g/4.7953 mol), acetone (161.55 g/2.7774 moles), potassium carbonate (94.03 g/0.6803 mol) and benzoquinone (0.76/7.03×10⁻³ mol). The reaction mixture was stirred while CTFE (78.92 g/0.6776 mol) was added subsurface in aliquots through a septum over two days with the temperature ranging from 16-21° C. An internal standard (α,α,α-trifluorotoluene) was added to reaction mixture to follow reaction by FNMR.

The reaction mixture was charged to a 5 L separatory funnel with 2 L of water and 1 L of dichloromethane and stirred for 10 minutes. The stirring was stopped and two immiscible layers formed after sitting for 15 minutes. The resulting layers were separated and the bottom organic layer was washed twice with 1 L of water. The organic layer was separated and the solvent stripped at reduced pressure to isolate the product. The amount of crude 2-Chloro-1,1,2-trifluoroethoxy methacrylate product isolated was 120.90 g. The product had a purity of 73 wt % and a yield of 58% by FNMR based on 2-Hydroxymethacrylate starting material.

The crude material was purified by column chromatography using a 2″×24″ column packed with silica gel. The ratio of silica to crude material was 15:1. The product was eluted with 10% ethyl acetate/n-hexane. The crude was purified in multiple batches. The combined purified product was 66.99 grams and was 97% pure by GC A %. The product was also confirm by GC/MS and LC/MS. The yield of purified product was 43% based on 2-Hydroxymethacrylate starting material.

¹⁹F NMR (CDCl₃): δ −88.26 ppm (F_{A}), −88.74 ppm (F_{B})*, (q of d of d, ²J_{Fa-Fb}=−141 Hz, ³J_{Fa-H}=3.5 Hz, ³J_{Fb-H}=4.7 Hz), δ −154.31 (Fe) (d of t, ³J_{F-F}=12 Hz, ²J_{F-H}=48 * The chemical shifts of F_{A} and F_{B} were calculated from the AB type quartet.

¹HNMR (CDCl₃): δ 1.95 ppm (d of d, 3H); δ 4.20 ppm (d of d of d, 2H); δ 4.40 (d of d of d, 2H); δ 5.60 (d of m 1H) δ 6.08 ppm (d of d of d, 1H, ²J_{H-F}=48, ³J_{H-Fa}=3.5 Hz, ³J_{H-Fb}=4.7 Hz); δ 6.10 ppm (d of m, 1H)

### Example 32 Reaction of 1,1,2-Trifluoro-2-chloroethylene (CTFE) with 2-Hydroxyethylmethacrylate (HEMA) in DMSO Solvent

A 250 ml four-neck (14/20) flask was placed on a magnetic stirrer and equipped with a digital thermometer and a dry-ice condenser with outlet connected to a nitrogen source. A pre-puncture septum was placed on the remaining neck. The reaction flask was charged with 2-Hydroxyethylmethacrylate (20.12 g/0.1546 mol), DMSO (116.85 g/1.4956 mol), potassium carbonate (21.84 g/0.1580 mol) and benzoquinone (0.06/5.55×10⁻⁴ mol). The reaction mixture was stirred while CTFE (18.81 g/0.1615 mol) was added subsurface in aliquots through a septum over three hours with the temperature ranging from 17−25° C. An internal standard (α,α,α-trifluorotoluene) was added to reaction mixture to follow reaction by FNMR.

The reaction mixture was combined with 700 ml of water and 200 ml of methylene chloride and stirred for 15 minutes. The resulting mixture was placed in a separatory funnel where two immiscible layers formed after sitting for 15 minutes. The resulting layers were separated and the bottom organic layer was washed twice with 200 ml of water. The organic layer was separated and the solvent stripped at reduced pressure to isolate the product. The amount of crude 2-Chloro-1,1,2-trifluoroethoxy methacrylate product isolated was 33.34 g. The product had a purity of 74 wt % and a yield of 64% by FNMR based on 2-Hydroxymethacrylate starting material.

The crude material was purified by short path distillation under a vacuum of approximately 1 torr. The amount of distilled product collected was 27.02 g. The distilled product had a purity of 80 wt % and a yield of 57% by FNMR based on 2-Hydroxymethacrylate starting material.

¹⁹F NMR (CDCl₃): δ −88.26 ppm (F_{A}), −88.74 ppm (F_{B})*, (q of d of d, ²J_{Fa-Fb}=−141 Hz, ³J_{Fa-H}=3.5 Hz, ³J_{Fb-H}=4.7 Hz), δ −154.31 (F_{c}) (d of t, ³J_{F-F}=12 Hz, ²J_{F-H}=48 * The chemical shifts of F_{A} and F_{B} were calculated from the AB type quartet.

¹HNMR (CDCl₃): δ 1.95 ppm (d of d, 3H); δ 4.20 ppm (d of d of d, 2H); δ 4.40 (d of d of d, 2H); δ 5.60 (d of m 1H) δ 6.08 ppm (d of d of d, 1H, ²J_{H-F}=48, ³J_{H-Fa}=3.5 Hz, ³J_{H-Fb}=4.7 Hz); δ 6.10 ppm (d of m, 1H)

1. A method of making a halogenated organic compound, comprising reacting an active hydrogen-containing organic compound selected from the group consisting of alcohols, primary amines, secondary amines and thiols with a halogenated olefin containing a carbon-carbon double bond, wherein at least one carbon of the carbon-carbon double bond is substituted with at least one substituent selected from the group consisting of halogens and halogenated alkyl groups, to produce the halogenated organic compound.

2. The method of claim 1, wherein the halogenated olefin contains one or more fluorine atoms.

3. The method of claim 1, wherein the halogenated organic compound is a halogenated heteroalkenyl-functionalized organic compound.

4. The method of claim 1, wherein the halogenated organic compound is a halogenated heteroalkyl-functionalized organic compound.

5. The method of claim 1, wherein the halogenated olefin has a fluorinated alkyl group substituted on one carbon of the carbon-carbon double bond.

6. The method of claim 1, wherein the halogenated olefin has a perfluorinated alkyl group substituted on one carbon of the carbon-carbon double bond.

7. The method of claim 1, wherein the halogenated olefin has a structure in accordance with formula (1):
CX¹X²═CX³X⁴ (1)
wherein X¹, X², X³ and X⁴ are independently selected from the group consisting of hydrogen (H), chlorine (Cl), fluorine (F), bromine (Br), iodine (I) and halogenated and non-halogenated C1-C20 alkyl groups, subject to the proviso that one or more of X¹, X², X³ and X⁴ is selected from the group consisting of chlorine (Cl), fluorine (F), bromine (Br), iodine (I) and halogenated alkyl groups.

8. The method of claim 1, wherein the halogenated olefin is selected from the group consisting of CFCl═CH₂, CH₂═CF₂, CFH═CH₂, CF₂═CHF, CF₃CF═CH₂, CF₂═CF₂, CF═CHCl, CF₃CCl═CH₂, CF₃CH═CHCl, CF₃CF═CFH, CF₃CH═CF₂, CF₃CF═CF₂, CF₃CH₂CF═CH₂, CF₃CH═CFCH₃, CF₃CF═CHCF₃, CF₃CCl═CHCF₃, CF₂HCH₂CF═CH₂, CF₂HCH₂CF═CHCl and CF₂HCH═CFCH₂Cl.

9. The method of claim 1, wherein the halogenated olefin is reacted with a phenolic compound.

10. The method of claim 1, wherein the halogenated olefin is reacted with an aliphatic alcohol.

11. The method of claim 1, wherein the halogenated olefin is reacted with an aliphatic polyalcohol.

12. The method of claim 1, wherein the halogenated olefin is reacted with a masked aliphatic polyalcohol which is an aliphatic polyol having a plurality of hydroxyl groups wherein at least one hydroxyl group is blocked and at least one hydroxyl group is a free hydroxyl group.

13. The method of claim 1, wherein the halogenated olefin is reacted with a primary or secondary amine.

14. The method of claim 1, wherein the halogenated olefin is reacted with a thiol.

15. The method of claim 1, wherein the reacting is carried out under basic conditions.

16. The method of claim 1, wherein the reacting is carried out in the presence of an inorganic base.

17. The method of claim 16, wherein the inorganic base is selected from the group consisting of alkali metal hydroxides and alkali metal salts of carbonic acid.

18. The method of claim 1, wherein the reacting is carried out in a liquid medium.

19. The method of claim 18, wherein the liquid medium is comprised of one or more organic solvents.

20. The method of claim 19, wherein the one or more organic solvents are selected from the group consisting of polar, non-protic organic solvents.

21. The method of claim 19, wherein the one or more organic solvents are polar, non-protic organic solvents having dielectric constants of from 2 to 190.

22. The method of claim 1, wherein the reacting is carried out in the presence of a phase transfer catalyst.

23. The method of claim 2, wherein the phenolic compound has structure Ar(OH)ₓ, wherein Ar is an optionally substituted aromatic moiety and x is an integer of 1 or more.

24. The method of claim 23, wherein x is 1, 2 or 3.

25. The method of claim 23, wherein Ar is selected from the group consisting of optionally substituted phenyl groups, optionally substituted naphthyl groups, and optionally substituted anthryl groups.

26. The method of claim 23, wherein Ar is an aromatic moiety substituted with one or more substituents selected from the group consisting of halogen, optionally substituted alkyl, optionally substituted cycloalkyl, optionally substituted heteroalkyl, optionally substituted heterocycloalkyl, optionally substituted alkoxy, optionally substituted aroxy, optionally substituted aryl, optionally substituted heteroaryl, cyano, optionally substituted carboxyl, sulfate and nitro.

27. The method of claim 1, wherein the active hydrogen-containing organic compound and the halogenated olefin are reacted at a temperature of from about 200° C. to about 200° C. for a time of from about 0.5 hours to about 120 hours.

28. The method of claim 1, wherein the active hydrogen-containing organic compound and the halogenated olefin are reacted in a stoichiometric ratio of (moles active hydrogen-containing organic compound)/x:moles halogenated olefin, wherein x=number of active hydrogens per molecule of the active hydrogen-containing organic compound, of from about 1:8 to about 8:1.

29. A trifluoropropenylether-substituted aromatic compound of formula (I):
Ar(OCR¹═CHR²)ₓ (I)
wherein Ar is an optionally substituted aromatic moiety, x is an integer of 1 or more, and either R¹ is CF₃ and R² is H or R¹ is H and R² is CF₃.

30. The trifluoropropenylether-substituted aromatic compound of claim 29, wherein x is 1, 2 or 3.

31. The trifluoropropenylether-substituted aromatic compound of claim 29, wherein Ar is selected from the group consisting of optionally substituted phenyl groups, optionally substituted naphthyl groups, and optionally substituted anthryl groups.

32. The trifluoropropenylether-substituted aromatic compound of claim 29, wherein Ar is an aromatic moiety substituted with one or more substituents selected from the group consisting of halogen, alkyl, cyano, sulfate and nitro.

33. The trifluoropropenylether-substituted aromatic compound of claim 29, wherein the trifluoropropenylether-substituted aromatic compound is selected from the group consisting of 4-chlorophenyl-3,3-3-trifluoropropenyl ether, 1,4-bis(3,3,3-trifluoropropenyloxy)benzene, 4-fluorophenyl-3,3,3-trifluoropropenyl ether, 4-methylphenyl-3,3,3-trifluoropropenyl ether, 3-cyanophenyl-3,3,3-trifluoropropenyl ether, 2-fluorophenyl-3,3,3-trifluoropropenyl ether, 3-nitrophenyl-3,3,3-trifluoropropenyl ether, 2,4-dichlorophenyl-3,3,3-trifluoropropenyl ether, 2-chloro-4-fluorophenyl-3,3,3-trifluoropropenyl ether, 4-(3,3,3-trifluoropropenyl)phenyl sulfate, 4-fluorophenyl-3,3,3-trifluoroprop-2-enyl ether, 3-nitrophenyl-3,3,3-trifluoroprop-2-enyl ether, 2-fluorophenyl-3,3,3-trifluoroprop-2-enyl ether, 4-methylphenyl-3,3,3-trifluoroprop-2-enyl ether, 4-chlorophenyl-3,3,3-trifluoroprop-2-enyl ether, 3-cyanophenyl-3,3,3-trifluoroprop-2-enyl ether, 1,4-bis(3,3,3-trifluoroprop-2-enyl)phenyl ether, 2,4-dichlorophenyl-3,3,3-trifluoroprop-2-enyl ether, 2-chloro-4-fluorophenyl-3,3,3-trifluoroprop-2-enyl ether, and 4-(3,3,3-trifluoroprop-2-enyl)phenyl sulfate, sodium salt.

34. A haloalkyl ether (meth)acrylate corresponding to general structure (I):
X¹X²HC—CX³X⁴—O—R—O—C(═O)—CR¹═CH₂ (I)
wherein R is an organic moiety, X¹, X², X³ and X⁴ are independently selected from hydrogen, halogen, alkyl or haloalkyl, subject to the proviso that at least one of X¹, X², X³ or X⁴ is halogen or a haloalkyl group, and R¹ is hydrogen or methyl or fluorine.

35. The haloalkyl ether (meth)acrylate of claim 34, wherein at least two of X¹, X², X³ or X⁴ are selected from the group consisting of halogens and haloalkyl groups.

36. The haloalkyl ether (meth)acrylate of claim 34, wherein at least two of X¹, X², X³ or X⁴ are selected from the group consisting of fluorine and fluoroalkyl groups.

37. The haloalkyl ether (meth)acrylate of claim 34, wherein at least one of X¹, X², X³ or X⁴ is fluorine or a fluoroalkyl group.

38. The haloalkyl ether (meth)acrylate of claim 34, wherein each of X¹, X², X³ and X⁴ is halogen or a haloalkyl group.

39. The haloalkyl ether (meth)acrylate of claim 34, wherein one of X¹, X², X³ or X⁴ is a C1-C8 haloalkyl group.

40. The haloalkyl ether (meth)acrylate of claim 34, wherein one of X¹, X², X³ or X⁴ is a C1-C8 fluoroalkyl group.

41. The haloalkyl ether (meth)acrylate of claim 34, wherein a) X¹ is chlorine and X², X³ and X⁴ are fluorine or b) X³ is chlorine and X¹, X² and X⁴ are fluorine.

42. The haloalkyl ether (meth)acrylate of claim 34, wherein R is an alkylene segment or a poly(oxyalkylene) segment.

43. The haloalkyl ether (meth)acrylate of claim 34, wherein R is an ethylene segment or a poly(oxyethylene) segment.

44. The haloalkyl ether (meth)acrylate of claim 34, wherein R is —[CH₂CH₂O]ₙ—CH₂CH₂— and n is 0 or an integer of from 1 to 10.

45. The haloalkyl ether (meth)acrylate of claim 34, wherein the moiety X¹X²HC—CX³X⁴—O—R—O— has a molecular weight not greater than 900 daltons.

46. The haloalkyl ether (meth)acrylate of claim 34, wherein R is a non-halogenated organic moiety.

47. The haloalkyl ether (meth)acrylate of claim 34, wherein R is an aliphatic organic moiety, optionally containing one or more oxygen atoms.

48. The haloalkyl ether (meth)acrylate of claim 34, wherein R is a saturated aliphatic organic moiety, optionally containing one or more ether oxygen atoms.

49-62. (canceled)
